(19) United States
(12) Patent Application Publication
(10) Pub. No.: US 2023/0312457 A1
(43) Pub. Date: Oct. 5, 2023

(54) LOW-MIGRATION HINDERED PHENOL ANTIOXIDANT COMPOUND, PREPARATION METHOD AND COMPOSITION

(71) Applicant: Jerry TSENG, TAIPEI (TW)

(72) Inventors: Jerry TSENG, TAIPEI (TW); Hai-Tao WEI, TAIPEI (TW)

(21) Appl. No.: 17/914,559

(22) PCT Filed: Mar. 24, 2021
(86) PCT No.: PCT/CN2021/082762

§ 371 Date: Sep. 26, 2022

(30) Foreign Application Priority Data
Mar. 26, 2020 (CN) 202010222099.5

(51) Int. Cl. C07C 69/732; C07C 323/56; C07C 69/734; C07C 235/34; C07D 493/10; C07C 243/32; C07D 251/70; C07D 251/32; C08K 5/134; C08K 5/1575; C08K 5/375; C08K 5/3492

(52) U.S. Cl. CPC C07C 69/732; C07C 69/734; C07C 235/34; C07C 243/32; C07C 323/56; C07D 251/32; C07D 251/70; C07D 493/10; C08K 5/1345; C08K 5/1575; C08K 5/34922; C08K 5/375

(57) ABSTRACT

Disclosed are a low-migration hindered phenol antioxidant compound, a preparation method and a composition. During production, processing or use, polymers experience degradation due to factors such as light, oxygen and heat. The oxidation resistance thereof is often increased by adding one or more common antioxidants, thereby inhibiting and delaying the rate if oxidative degradation thereof. The structures of traditional hindered phenolic antioxidant compounds migrate in polymers. The hindered phenolic antioxidant compound of the present invention has more hindered phenol units, and can achieve the objectives of low extraction and low migration.

### BACKGROUND

### Technology Field

The invention relates to a low-migration hindered phenol antioxidant compound, a preparation method and a composition.

### Description of Related Art

Polymer materials are often degraded by light, oxygen, heat and other factors during production, processing and use, resulting in a decrease in the physical and chemical properties of polymer materials. Therefore, polymer materials often increase their antioxidant capacity by adding one or more antioxidants. And thus inhibit or delay its oxidative degradation thereafter prolong its service life.

Among them, hindered phenolic compounds are one of the most important antioxidant compounds. Hindered phenolic antioxidants have been widely used in improving the thermal oxidative aging resistance of polymers.

However, the traditional hindered phenolic antioxidant compounds will migrate from the inside of the polymer and seriously affect the performance.

Traditional hindered phenol antioxidants include, for example, 2,4,6-tri-tert-butylphenol (AO333), dibutylhydroxytoluene (BHT), and Irganox 1076. Because of their strong volatility, they are easy to diffuse from the polymer and migrate to the surface of the polymer, and eventually the content of antioxidants in the polymer disappears, which seriously affects the performance. And antioxidants enter the environment, destroy the ecology, and also harm human health.

Therefore, it is of great significance to design hindered phenol antioxidants with migration resistance. One of the current solutions to these problems is to develop multi-unit hindered phenol antioxidants to delay migration. For example Irganox 245 is a 2 unit hindered phenolic antioxidant, e.g. Irganox 1330 is a 3 unit hindered phenolic antioxidant, and e.g. Irganox 1010 is a 4 unit hindered phenolic antioxidant. However, simply increasing the molecular weight of hindered phenol antioxidants does not necessarily achieve both anti-migration and antioxidant properties. How to develop better multi-unit hindered phenol antioxidants with anti-migration properties has become the goal of the industry.

### SUMMARY

In order to solve the deficiencies of the prior art, the present invention provides a low-migration hindered phenol antioxidant compound, a preparation method and a composition, so that the ratio of “hindered phenol unit/molecular weight” is maintained in an optimal range. The method includes optimizing (number of hindered phenol units/molecular weight) so that the ratio of (number of hindered phenol units/molecular weight) after optimization and before optimization is greater than 1. That is, the newly added antioxidant unit will not increase the molecular weight too much. For example, the ratio of number of hindered phenol units/molecular weight of compound 10 of the present invention (Example 10) and Eunox 1035 (Example 60) is (4/966): (2/642) =1.33 (>1).

Surprisingly, such a design greatly improves the retention of antioxidants in the resin, that is, it solves the disadvantage of easy migration of traditional hindered phenolic antioxidants.

Technical scheme: in order to solve the above-mentioned technical problems, the technical scheme adopted in the present invention is:

A first aspect provides a compound of formula (I) or a salt thereof,

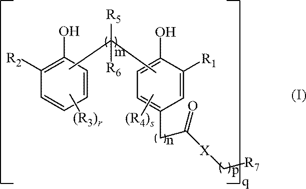

wherein, R₁—R₇ are substituent groups, and R₁—R₂ are each independently selected from alkyl, phenyl, benzyl, cumyl, C₁-C₁₂ containing C₁-C₁₂, preferably C₁-C₁₂ sulfane, C₁-C₁₂ methylene C₁-C₁₂sulfane; R₃—R4 are selected from hydrogen, C₁-C₆-containing alkyl, phenyl, benzyl, cumyl, C₁-C₁₂ sulfane, C₁-C₂ methylene C₁-C₁₂ sulfane; R₅—R₆ are each independently selected from hydrogen, hydroxyl, halogen, carboxyl, C₁-C₆ alkyl, carbonyl, acyl, ester, C₁-C₆ alkylamino, C₁-C₆ alkoxy, phenyl, or R₅ —R₆ is combined into a ketone group. Preferably, R₁—R₂ are each independently selected from C₁-C₅ -containing alkyl, phenyl, benzyl, and cumyl; R₃—R₄ are each independently selected from hydrogen, hydroxyl, C₁-C₅-containing alkyl, carbonyl, acyl group, C₁-C₅ alkylamino group, C₁-C₅ alkoxy group; R₅—R₆ are each independently selected from hydrogen, hydroxyl, C₁-C₅ -containing alkyl group, and phenyl group. Particularly preferably, R₁—R₂ are each independently selected from methyl, tert-butyl, and cumyl; R₃—R₄ are each independently selected from hydrogen, hydroxyl, methyl, and tert-butyl; R₅—R₆ are each independently selected from hydrogen, hydroxyl, alkyl containing C₁-C₄; R₇ is a q-valent group. Preferably, R₇ includes a bond, hydrogen, unsubstituted or substituted heteroatom, unsubstituted or substituted carbon or carbon chain, unsubstituted or substituted carbon chain interrupted by oxygen or sulfur or nitrogen, carbon ring, heterocycle.

More preferably, the carbon is primary to quaternary carbon, the carbon chain is a C₁-C₂₀ carbon chain, a carbon chain interrupted by oxygen or sulfur or nitrogen, which may be a non-polymeric C₁-C₂₀ heterocarbon chain or a chain comprising multiple polymerized units, such as polyethylene glycol. Preferably, the carbocycle is a five to seven membered monocyclic ring, and the heterocycle is a five to seven membered monocyclic ring containing oxygen or sulfur or nitrogen.

More preferably, R₇ includes H, a bond,,(C)ₐ(CH)_{b}(CH₂)_{c}(CH₃)_{d} (wherein, the order between (C)ₐ and (CH)_{b} and (CH2)_{c} and (CH3)_{d} can be interchanged, a~d are not 0 at the same time, a~d = 0~18), (CH₂CH₂O)ₜ H, (CH₂CH₂O)ₜOCH₃, (CH)_{q-2}(CH₂)_{1~10}(CH₃)_{1∼4}. Preferably, (CH)_{q2}(CH₂)_{1~10}(CH₃)_{1~3},, wherein the order among (CH)_{q-2},(CH₂)_{1~10} and (CH₃)_{1∼3} can be interchanged, S, SH, O, OH, N, NH, NHR₈, P, Ca, Mg, Zn, Na, K, -(CHR₈)_{1∼18}-, -(CH)_{q-2}(CH₂)_{1∼18}-, where the order between (CH)_{q-2} and (CH₂)_{1∼18} can be interchanged, -(C=O)₁₋₄-, -(CHR₈) ᵤ -, -(C=O)₁₋₄(CHR₈) ᵤ -, where the order among (C=O)₁₋₄, (CHR₈)ᵤ (C=O)₁₋₄ and (CHR₈)ᵤ can be interchanged, -(CHR₈)ᵤS₁₋₄(CHR₈)u-, -(CHR₈)ᵤO₁₋₄(CHR₈)u-, —(CH₂CH₂O)ₜCH₂CH₂—,

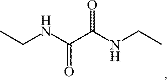

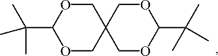

triazines, melamines, unsubstituted or substituted phenyl or benzyl, C₁-C₈ cycloalkyl; q≧ a+b+c+d; t=1-20, u=1-20. Wherein the order between (CH)_{q-2}and (CH₂) _{1∼18} can be interchanged, preferably, -(CH)_{q-2}(CH₂)_{1∼18}- is —(CH₂) ₁(CH)(CH₂)₁— or —(CH₂)₂(CH)(CH₂)₂—. Preferably, (C)a(CH)b(CH2)c(CH3)d, is (C)a(CH)_{b}(CH₂)_{c}(CH₃)_{d}, is C, CH, CH₂, CH₃, a~d = 0-8, more preferably, a~d=0-4. Preferably, t=1-10. u=1-10. More preferably, t=1-5. u=1-5.

X is carbon or a heteroatom, preferably, selected from N, NH, NHR₈, O, S, CH₂, CHR₈, R₈ is selected from H, OH, C₁-C₆ containing alkyl, more preferably, X is selected from NH, O, CH₂, particularly preferably, X=NH or O.

m=0-5, n=0-5, p=0-18, q=1-8, r=0-3, s=0-2. Preferably, m=0-2, n=0-2, p=0-18, q=1-6, r=0-1, s=0-1. More preferably, m=1, n=2, q=1-4, r=1, s=0.

Particularly preferably, formula (I) is the following structure:

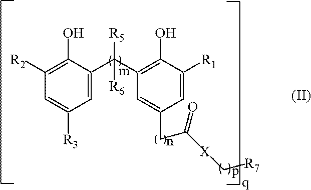

R₁—R₇, m, n, X, p, r are as defined above.

Particularly preferably, formula (I) is the following structure:

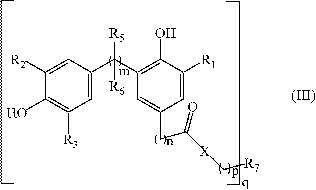

R₁—R₇, m, n, X, p, q, r are as defined above. When R7 ═—(CH₂CH₂O)ₜ CH₂CH₂—, t>1.

Particularly preferably, formula (I) is the following structure:

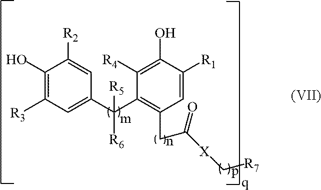

R₁—R₇, m, n, X, p, q, r are as defined above.

The preparation method of compound shown in formula (I), is characterized in that, comprises an esterification or transesterification reaction as follows:

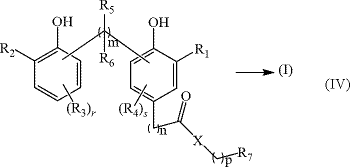

Wherein R₁—R₆, n, r, s are as defined above; —X(CH₂)p, R₇ is OH or a leaving group. Preferably, the leaving group is OCH₃ or halogen. Wherein compound (IV), is synthesized by following esterification or transesterification reaction formula:

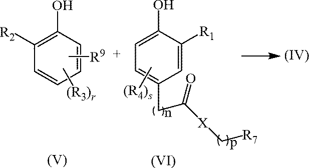

Wherein, R₉ is a group on a benzene ring or a non-benzene ring that can produce a Friedel-Crafts alkylation or acylation reaction, and when R₉ is a group on a benzene ring, it includes halogen, C₁— C₈ haloalkyl, haloacyl, C₁-C₈ haloacyl, C₁-C₈ alkenyl. When R₉ is a group other than a benzene ring, it includes a C₁-C₈ alkyl aldehyde or ketone.

For the reaction of the present invention, any esterification or transesterification catalyst can be used, preferably aluminum triisopropoxide or a tin compound, especially dibutyltin diacetate. Examples of catalysts useful in the practice of this invention include stannous octoate, stannous oxalate, dibutyltin dilaurate, dioctyl dilaurate, dibutyl dioctyl-2-ethylhexanoate, tetra Isopropyl, tetrabutyl titanate, tetra-2-ethylhexyl titanate, dibutyl difuryl mercaptan, dibutyl diindolyl octyl mercaptoacetate, dibutyl tin dilaurate, Dibutyltin oxide, butylstannic acid, etc.

The compounds of formula (I) of the present invention, which can be used in compositions, provide antioxidant function. The composition can be applied to various organic materials such as, but not limited to, polyols or polyurethanes. Polyols will release a lot of heat during the subsequent production of polyurethane foam, causing yellowing. If general antioxidants are added, the antioxidants themselves will precipitate on the surface, which will also cause yellowing. The hindered phenol antioxidant of the present invention is suitable for various materials. Taking nylon-6 resin as an example, it is 0.1 to 5 parts by weight in 100 parts by weight. The hindered phenolic antioxidant of the present invention can be used together with a phosphite antioxidant, and the mixing weight ratio of the hindered phenolic antioxidant and the phosphite antioxidant is preferably 1:4 to 1:1. The hindered phenolic antioxidant of the present invention may also be used in combination with other stabilizers, such as ultraviolet absorbers, hindered amines, and the like.

### DETAILED DESCRIPTION OF THE DISCLOSURE

The present invention will be further described below in connection with the examples. The following examples are only used to more clearly illustrate the performance of the present invention, and should not be limited to the following examples.

The hindered phenolic antioxidants of the present invention are specifically represented by examples, but are not limited to the compounds of the examples. Wherein R7 is a linking structure, as shown in Table 1.

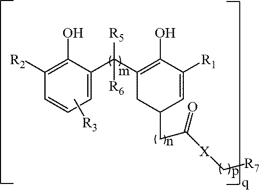

TABLE 1

| Ex. | R₁ | R₂ | R₃ | R₅R₆ | R₇ | X | m | n | p | q |
|---|---|---|---|---|---|---|---|---|---|---|
| 1 | t-Bu | t-Bu | 5-tBu | 2H | CH3 | O | 1 | 2 | 0 | 1 |
| 2 | t-Bu | t-Bu | 5-tBu | =O | CH3 | 0 | 1 | 2 | 0 | 1 |
| 3 | t-Bu | t-Bu | 5-tBu | H,OH | CH3 | 0 | 1 | 2 | 0 | 1 |
| 4 | t-Bu | t-Bu | CH3 | CH3 | CH3 | 0 | 1 | 2 | 0 | 1 |
| 5 | t-Bu | CH3 | 5-tBu | 2H | CH3 | 0 | 1 | 2 | 0 | 1 |
| 6 | t-Bu | t-Bu | 5-tBu | 2H | C8H17 | 0 | 1 | 2 | 0 | 1 |
| 7 | t-Bu | t-Bu | 5-tBu | 2H | C18H37 | 0 | 1 | 2 | 0 | 1 |
| 8 | t-Bu | t-Bu | 5-tBu | 2H | (CH2CH2O)3CH3 | 0 | 1 | 2 | 0 | 1 |
| 9 | t-Bu | t-Bu | 5-tBu | 2H | (CH2CH2O)3H | 0 | 1 | 2 | 0 | 1 |
| 10 | t-Bu | t-Bu | 5-tBu | 2H | S | O | 1 | 2 | 2 | 2 |
| 11 | t-Bu | t-Bu | 5-tBu | 2H | (CH2CH2O)2CH2CH2 | O | 1 | 2 | 0 | 2 |
| 12 | t-Bu | t-Bu | 5-tBu | 2H | (CH2)6 | NH | 1 | 2 | 0 | 2 |
| 13 | t-Bu | t-Bu | 5-tBu | 2H | (CH2)6 | O | 1 | 2 | 0 | 2 |
| 14 | t-Bu | t-Bu | 5-tBu | 2H | (NHC=O)2 | O | 1 | 2 | 2 | 2 |
| 15 | t-Bu | t-Bu | 5-tBu | 2H | 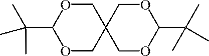 | O | 1 | 2 | 0 | 2 |
| 16 | t-Bu | t-Bu | 5-tBu | 2H | a bond | NH | 1 | 2 | 0 | 2 |
| 17 | t-Bu | t-Bu | 5-tBu | 2H | C | O | 1 | 2 | 1 | 4 |
| 18 | t-Bu | t-Bu | 5-tBu | 2H | 1,3,5- triazine | O | 1 | 2 | 0 | 3 |
| 19 | t-Bu | t-Bu | 5-tBu | 2H | melamine | O | 1 | 2 | 2 | 3 |
| 20 | t-Bu | t-Bu | 5-tBu | 2H | Trimethyl benzene | O | 1 | 2 | 2 | 3 |
| 21 | t-Bu | t-Bu | 5-tBu | 2H | Benzene | O | 1 | 2 | 1 | n |
| 22 | t-Bu | t-Bu | 5-tBu | 2H | CH2CHCH2 | O | 1 | 2 | 0 | 3 |
| 23 | t-Bu | t-Bu | 5-tBu | 2H | Ca | O | 1 | 2 | 0 | 2 |
| 24 | cumyl | cumyl | 5-tBu | 2H | CH3 | O | 1 | 2 | 0 | 1 |
| 25 | CH3 | CH2SCH2H25 | 5-tBu | 2H | CH3 | O | 1 | 2 | 0 | 2 |

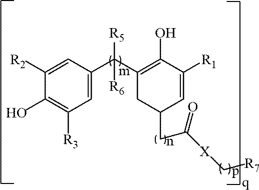

TABLE 2

| | | | | | | | | | | |
|---|---|---|---|---|---|---|---|---|---|---|
| 26 | t-Bu | t-Bu | 5-tBu | 2H | CH3 | O | 1 | 2 | 0 | 1 |
| 27 | t-Bu | t-Bu | 5-tBu | =O | CH3 | 0 | 1 | 2 | 0 | 1 |
| 28 | t-Bu | t-Bu | 5-tBu | H, OH | CH3 | 0 | 1 | 2 | 0 | 1 |
| 29 | t-Bu | t-Bu | 5-tBu | CH3 | CH3 | 0 | 1 | 2 | 0 | 1 |
| 30 | t-Bu | CH3 | 5-tBu | 2H | CH3 | 0 | 1 | 2 | 0 | 1 |
| 31 | t-Bu | t-Bu | 5-tBu | 2H | C8H17 | 0 | 1 | 2 | 0 | 1 |
| 32 | t-Bu | t-Bu | 5-tBu | 2H | C18H37 | 0 | 1 | 2 | 0 | 1 |
| 33 | t-Bu | t-Bu | 5-tBu | 2H | (CH2CH2O)CH2CH2 | 0 | 1 | 2 | 0 | 1 |
| 34 | t-Bu | t-Bu | 5-tBu | 2H | (CH2CH2O)2CH2CH2 | 0 | 1 | 2 | 0 | 1 |
| 35 | t-Bu | t-Bu | 5-tBu | 2H | (CH2CH2S)CH2CH2 | 0 | 1 | 2 | 0 | 1 |
| 36 | t-Bu | t-Bu | 5-tBu | 2H | (CH2CH2O)9CH2CH2 | O | 1 | 2 | 0 | 2 |
| 37 | t-Bu | t-Bu | 5-tBu | 2H | (CH2)6 | NH | 1 | 2 | 0 | 2 |
| 38 | t-Bu | t-Bu | 5-tBu | 2H | (CH2)6 | O | 1 | 2 | 0 | 2 |
| 39 | t-Bu | t-Bu | 5-tBu | 2H | (NHC=O)2 | O | 1 | 2 | 2 | 2 |
| 40 | t-Bu | t-Bu | 5-tBu | 2H | 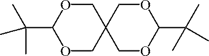 | O | 1 | 2 | 0 | 2 |
| 41 | t-Bu | t-Bu | 5-tBu | 2H | A bond | NH | 1 | 2 | 0 | 2 |
| 42 | t-Bu | t-Bu | 5-tBu | 2H | C | O | 1 | 2 | 1 | 4 |
| 43 | t-Bu | t-Bu | 5-tBu | 2H | 1,3,5- triazine | O | 1 | 2 | 0 | 3 |
| 44 | t-Bu | t-Bu | 5-tBu | 2H | melamine | O | 1 | 2 | 2 | 3 |
| 45 | t-Bu | t-Bu | 5-tBu | 2H | benzene | O | 1 | 2 | 2 | 3 |
| 46 | t-Bu | t-Bu | 5-tBu | 2H | CH2CHCH2 | O | 1 | 2 | 0 | 3 |
| 47 | cumyl | cumyl | 5-tBu | 2H | CH3 | O | 1 | 2 | 0 | 1 |
| 48 | cumyl | cumyl | 5-tBu | 2H | (CH2)6 | O | 1 | 2 | 0 | 2 |
| 49 | CH3 | CH2SC12H25 | 5-tBu | 2H | CH3 | O | 1 | 2 | 0 | 1 |
| 50 | CH3 | CH2SC12H25 | 5-tBu | 2H | (CH2)4 | O | 1 | 2 | 0 | 2 |

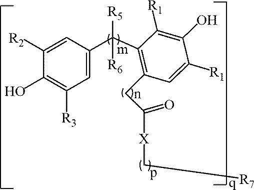

TABLE 3

| Ex. | R₁,R₄ | R₂ | R₃ | R₅R₆ | R₇ | X | m | n | p | q |
|---|---|---|---|---|---|---|---|---|---|---|
| 51 | t-Bu | t-Bu | 5-tBu | 2H | CH3 | O | 1 | 2 | 0 | 1 |
| 52 | t-Bu | t-Bu | 5-tBu | =O | CH3 | 0 | 1 | 2 | 0 | 1 |
| 53 | t-Bu | t-Bu | 5-tBu | H,OH | CH3 | 0 | 1 | 2 | 0 | 1 |
| 54 | t-Bu | t-Bu | 5-tBu | acetyl | CH3 | 0 | 1 | 2 | 0 | 1 |
| 55 | t-Bu | CH3 | 5-tBu | Bz | CH3 | 0 | 1 | 2 | 0 | 1 |
| 56 | t-Bu | t-Bu | 5-tBu | 2H | C8H17 | 0 | 1 | 2 | 0 | 1 |
| 57 | t-Bu | t-Bu | 5-tBu | 2H | (CH2CH2O)2CH2CH2 | 0 | 1 | 2 | 0 | 1 |
| 58 | t-Bu | t-Bu | 5-tBu | 2H | a bond | N | 1 | 2 | 0 | 2 |
| 59 | t-Bu | t-Bu | 5-tBu | 2H | C | 0 | 1 | 2 | 1 | 4 |

### Example 1

### Methyl 3-(3-tert-butyl-5-(3,5-di-tert-butyl-2-hydroxybenzyl)-4-hydroxyphenyl)propanoate

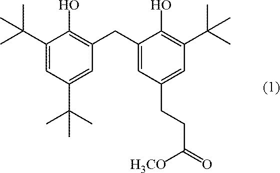 [SMILES: COC(=O)CCc1cc(Cc2cc(C(C)(C)C)cc(C(C)(C)C)c2O)c(O)c(C(C)(C)C)c1]

Take 25.4 g of 2,4-di-tert-butyl-6-chloromethylphenol (compound 1.1) and 23.6 g of methyl 3-(3-(tert-butyl)-4-hydroxyphenyl) propionate (CAS No 36837-50-0, mp=60° C.), dissolved in 200 mL of dry CH₂Cl₂, stirred at room temperature under nitrogen, and added 14 g of AlCl₃ to continue stirring. The reaction was monitored by TLC during which AlCl₃ was supplemented. After the reaction was completed, the mixture was poured into 200 mL of ice water, stirred, and extracted three times with CH2Cl2. The extract phases were combined, washed successively with 1% dilute hydrochloric acid and brine, and dried over anhydrous sodium sulfate. The solvent was evaporated to dryness, and the obtained residue was purified by column chromatography to obtain compound (1). MS(m/z) =454.3. H1-NMR(CDCl3). The chemical shift is 4.0, (aromatic carbon-CH₂-aromatic carbon) is a new peak, indicating that compound (1) is synthesized.

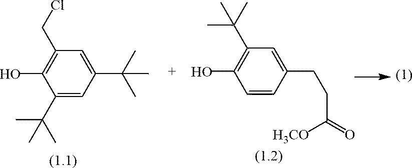

2,4-di-tert-butyl-6-chloromethylphenol (compound 1.1) preparation method: in a 50ml reaction flask, 3 g of 2,4-di-tert-butylphenol (96-76-4), 0.6 g of Paraformaldehyde, 20 grams of acetic acid, and 3 grams of 35% hydrochloric acid were added. The temperature was raised to 60° C., the reaction was incubated for 10 hours, and samples were taken to monitor the reaction. Cooling, washing and drying to obtain compound 1.1. Melting point: 62° C.

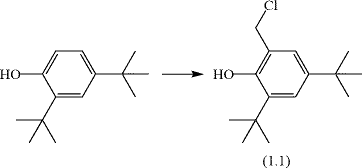

### Example 2

### 3-(3-Tert-butyl-5-(3,5-di-tert-butyl-2-hydroxybenzoyl)-4-hydroxyphenyl)pro panoate (2)

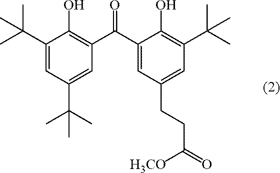 [SMILES: COC(=O)CCc1cc(C(=O)c2cc(C(C)(C)C)cc(C(C)(C)C)c2O)c(O)c(C(C)(C)C)c1]

According to the method of Example 1, but using 3,5-di-tert-butyl-2-hydroxybenzoyl chloride instead of 2,4-di-tert-butyl-6-chloromethylphenol (compound 1.1), compound (2) was obtained,reduction to obtain compound (1). Compound (2): MS (m/z) = 468.3. C13-NMR (CDCl3), chemical shift 199.1, (aromatic-C(O)-aromatic) is a new peak, indicating that compound (2) was synthesized.

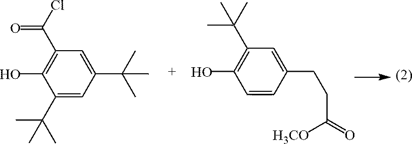

### Example 3

### Methyl 3-(3-tert-butyl-5-((3,5-di-tert-butyl-2-hydroxyphenyl) (hydroxy)methyl)-4-hydroxyphenyl)propanoate

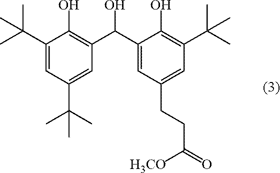 [SMILES: COC(=O)CCc1cc(C(O)c2cc(C(C)(C)C)cc(C(C)(C)C)c2O)c(O)c(C(C)(C)C)c1]

A mixture of 100 ml of ethanol and 10 g of compound (2) was cooled with ice water, 7.4 g of NaBH₄ was added, and the mixture was stirred for 1 hr to complete the reaction. The reaction was neutralized with glacial acetic acid and then concentrated in vacuo. The concentrate was partitioned between CH₂Cl₂ and water, then the organic phase was separated, washed with saturated brine, dried over anhydrous Na₂SO₄ and concentrated. Compound (3) is obtained. MS(m/z) = 470.3. H1-NMR (CDCl₃), a peak at chemical shift 6.2 (aromatic carbon-HCOH-aromatic carbon) was newly formed, indicating that compound (3) was synthesized.

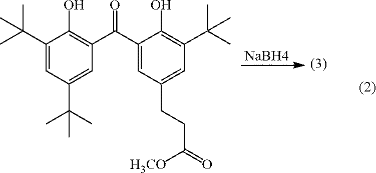

### Example 4

### Methyl 3-(3-(tert-butyl)-5-(1-(3-tert-butyl-5-methyl-2-hydroxyphenyl)ethyl)-4-hydroxyphenyl)propanoate

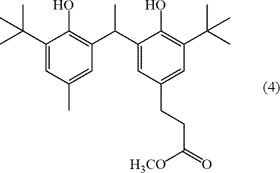 [SMILES: COC(=O)CCc1cc(C(C)c2cc(C)cc(C(C)(C)C)c2O)c(O)c(C(C)(C)C)c1]

According to the method of Example 1, except that acetaldehyde is used instead of polyoxymethylene, and 2-tert-butyl-4-methylphenol is used instead of 2,4-di-tert-butylphenol, the compound (4) is obtained by chromatographic separation. MS(m/z) = 426.3.

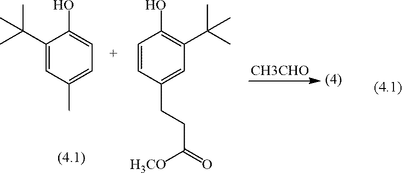

### Example 5

### Methyl 3-(3-(tert-butyl)-5-(3-methyl-5-(tert-butyl)-2-hydroxybenzyl)-4-hydroxyphenyl)propanoate

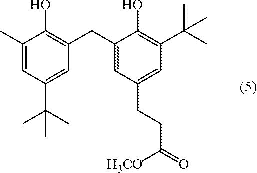 [SMILES: COC(=O)CCc1cc(Cc2cc(C(C)(C)C)cc(C)c2O)c(O)c(C(C)(C)C)c1]

According to the method of Example 1, but using 2-(chloromethyl)-4-(tert-butyl)-6-methylphenol instead of 2,4-di-tert-butylphenol, compound (5) is obtained. MS(m/z) = 412.3.

### Example 6

### Octyl 3-(3-(tert-butyl)-5-(3,5-di-tert-butyl-2-hydroxybenzyl)-4-hydroxyphenyl) propanoate

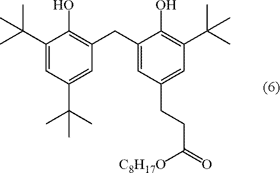

22.7 g of the compound of formula (1) prepared in Example 1 was mixed with 10 g of octanol (ExxonMobil Chemical) and 0.2 g of aluminum triisopropoxide (in toluene). The reaction mixture was stirred and heated to 85° C. under nitrogen atmosphere, and the resulting methanol was condensed and removed by vacuum. The reaction was monitored, and when the reaction was complete, aqueous citric acid (50%) was added and stirring continued for 20 minutes. Then, water was added andstirred for 20 minutes at 75° C. The organic phase was separated and washed twice with brine, then dried over sodium sulfate. Then toluene and excess octanol were distilled off under reduced pressure, and the residue was dried in vacuo. Separation by chromatography gave compound (6). MS(m/z) = 552.4.

### Example 7

### Octadecyl 3-(3-tert-butyl-5-(3,5-di-tert-butyl-2-hydroxybenzyl)-4-hydroxyphenyl)propanoate

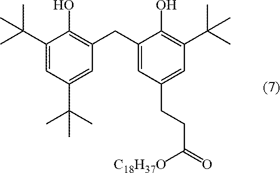

Following the procedure of Example 6, but substituting stearyl alcohol for octanol, compound (7) was obtained. MS(m/z)=692.6.

### Example 8

### 2,5,8,11,14,17,20-Heptoxapolysaccharide-22-yl-3-(3-(tert-butyl)-5-(3,5-di-tert-butyl-2 -hydroxybenzyl) yl)-4-hydroxyphenyl)propionate

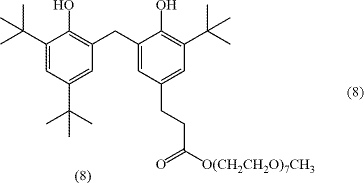

In the same method as in Example 7, Methoxypolyethylene glycol 350 wasused instead of octanol, and the product was purified by GPC to obtain compound (8).

### Example 9

### 2-Hydroxyethoxy)ethoxy)ethyl-3-(3-(tert-butyl)-5-(3,5-di-tert-butyl-2-hydroxyb enzyl)- 4-Hydroxyphenyl)propionate

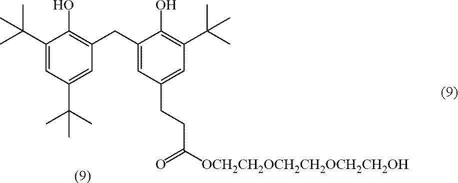

According to the method of Example 6, the molar ratio of compound (1) and glycols is controlled to be more than 2:1. 100 g of compound (1) and 12.2 g of 2,2′-thiodiethanol were used. Compound (10) is obtained. MS(m/z)=966.6.

### Example 10

### Thiobis(ethane-2,1-diyl)bis(3-(3-(tert-butyl)-5-(3,5-di-tert-butyl-2-hydroxybenzyl)-4-hydroxybenzene base) propionate) (10)

According to the method according to Example 6, the molar ratio of compound (1) and glycols is controlled to be 2:1 or more. 100 g of compound (1) and 12.2 g of 2,2′-thiodiethanol were used. Compound (10) is obtained. MS(m/z)=966.6.

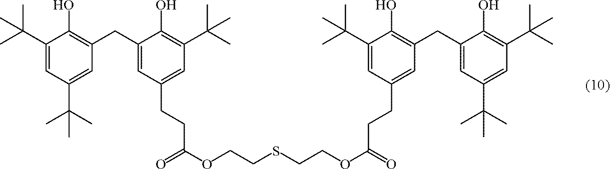 [SMILES: CC(C)(C)c1cc(Cc2cc(CCC(=O)OCCSCCOC(=O)CCc3cc(Cc4cc(C(C)(C)C)cc(C(C)(C)C)c4O)c(O)c(C(C)(C)C)c3)cc(C(C)(C)C)c2O)c(O)c(C(C)(C)C)c1]

### Example 11

### 2-((3-(3-(Tert-butyl)-5-(3,5-di-tert-butyl-2-hydroxybenzyl)-4-hydroxyphenyl)pro pionyl)oxy (yl)ethoxy)ethoxy)3-(3-(3,5-di-tert-butyl-2-hydroxybenzyl)-4-hydroxy-5-methylphen yl)propionate

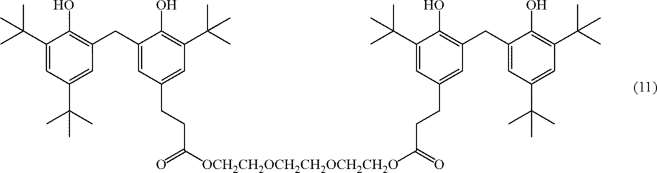

According to the method of Example 10, but using compound (1) and triethylene glycol. Compound (11) is obtained. MS(m/z) = 994.7.

### Example 12

### N,N′-(Propane-1,3-diyl)bis(3-(3-tert-butyl-5-(3,5-di-tert-butyl-2-hydroxybenzyl)-4-hy droxyphenyl) ) propionamide)

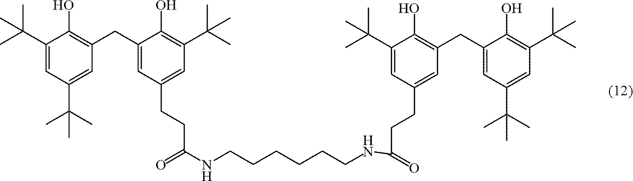 [SMILES: CC(C)(C)c1cc(Cc2cc(CCC(=O)NCCCCCCNC(=O)CCc3cc(Cc4cc(C(C)(C)C)cc(C(C)(C)C)c4O)c(O)c(C(C)(C)C)c3)cc(C(C)(C)C)c2O)c(O)c(C(C)(C)C)c1]

Take compound (1) hydrolyzate, 3-(3-(tert-butyl)-5-(3,5-di-tert-butyl-2-hydroxybenzyl)-4-hydroxyphenyl)propionicacid (compound 19- 1), 66 g (about 1.5 moles), 27 g (about 2.25 moles) of thionyl chloride, react at 90° C. for 3 hours, and evaporate the excess thionyl chloride under reduced pressure. 3-(3-(tert-butyl)-5-(3,5-di-tert-butyl-2-hydroxybenzyl)-4-hydroxyphenyl)propionyl chloride) (compound 12.2) was obtained. After being cooled to 60° C., add toluene 100 g, and stir.

A mixed solution consisting of 5.8 g (0.5 mol) of hexamethylene diamine, 10 g (1.25 mol) of pyridine and 50 g of toluene was added dropwise, and the temperature was controlled to be less than 60° C. After dripping, be warming up to 85° C., react 2 hours. After washing with water, it was dried, and the solvent was evaporated. Chromatography gave compound (12). MS(m/z) = 960.7.

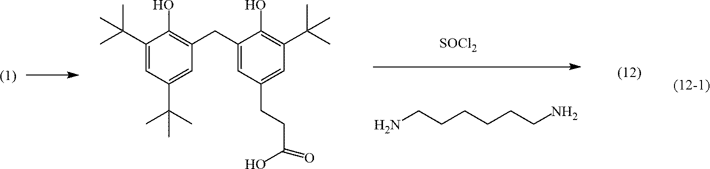

Hydrolysis of compound (1): 45.4 g of compound (1), 100 ml of methanol, were stirred under nitrogen. 22 ml of 30% NaOH solution were started dropwise at 60° C. After the dropwise addition, slowly heated to 65° C. for 4 h, neutralized by adding 160 mL of 2 N dilute hydrochloric acid, stirred for 2 h, washed with water to neutrality, and dried to obtain compound (1) free acid 3-(3-(tert. butyl)-5-(3,5-di-tert-butyl-2-hydroxybenzyl)-4-hydroxyphenyl)propionic acid) (compound 12-1).

### Example 13

### 1,6-Diylbis(3-(3-tert-butyl-5-(3,5-di-tert-butyl-2-hydroxybenzyl)-4-hydroxyphenyl)pr opionate hexyl)

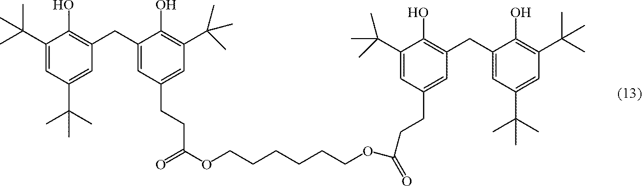 [SMILES: CC(C)(C)c1cc(Cc2cc(CCC(=O)OCCCCCCOC(=O)CCc3cc(Cc4cc(C(C)(C)C)cc(C(C)(C)C)c4O)c(O)c(C(C)(C)C)c3)cc(C(C)(C)C)c2O)c(O)c(C(C)(C)C)c1]

According to the method according to Example 10, but using compound (1) and hexanediol. Compound (13) is obtained. MS(m/z) = 947.6.

### Example 14

### Bis(ethane-2,1-diyl)bis(3-(3-tert-butyl-5-(3,5-di-tert-butyl-2-hydroxybenzyl))-4-hydro xyphenyl)propane Acid (oxalyl bis(aza-diyl)) ester

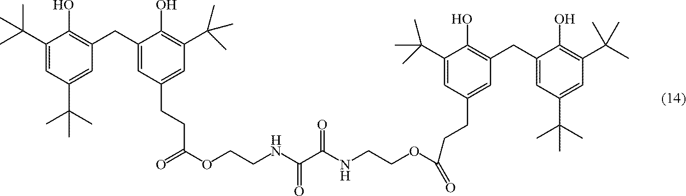 [SMILES: CC(C)(C)c1cc(Cc2cc(CCC(=O)OCCNC(=O)C(=O)NCCOC(=O)CCc3cc(Cc4cc(C(C)(C)C)cc(C(C)(C)C)c4O)c(O)c(C(C)(C)C)c3)cc(C(C)(C)C)c2O)c(O)c(C(C)(C)C)c1]

According to the method according to Example 10, but using compound (1) and N.N′-dihydroxyethyloxamide (1871-89-2, mp=168° C.). Compound (14) isobtained. MS(m/z) = 1020.6.

### Example 15

### (2,4,8,10-Tetraoxaspiro[5.5]undecane-3,9-diyl)bis(2-methylpropane-2,1-diyl)bis(3-(3-(tert-butyl)-5-(3,5-di-tert-butyl-2-hydroxybenzyl)-4-hydroxyphenyl)propionate)

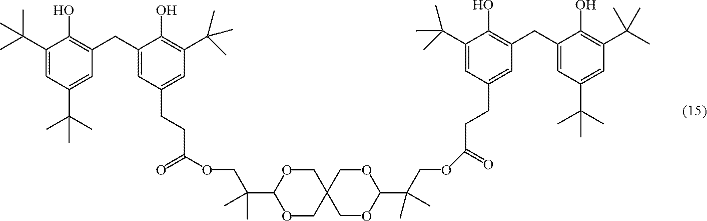 [SMILES: CC(C)(C)c1cc(Cc2cc(CCC(=O)OCC(C)(C)C3OCC4(CO3)COC(C(C)(C)COC(=O)CCc3cc(Cc5cc(C(C)(C)C)cc(C(C)(C)C)c5O)c(O)c(C(C)(C)C)c3)OC4)cc(C(C)(C)C)c2O)c(O)c(C(C)(C)C)c1]

Compound (1) was reacted with spiroethylene glycol according to the method according to Example 10. Compound (15) is obtained. MS(m/z) = 1148.8. Spirocyclic ethylene glycol, is an industrial raw material 2,2′-(2,4,8,10-tetraoxaspiro[5.5]undecane-3,9-diyl)bis(2-methylpropane-1 - alcohol) (mp=202° C.).

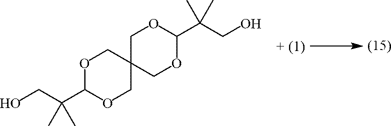

### Example 16

### 3-tert-butyl-5-(3,5-di-tert-butyl-2-hydroxybenzyl)-4-hydroxyphenyl)-N′-tert-butyl-5-(3,5-Di-tert-butyl-2-hydroxybenzyl)-4-hydroxyphenyl)propionyl)propanehydrazide

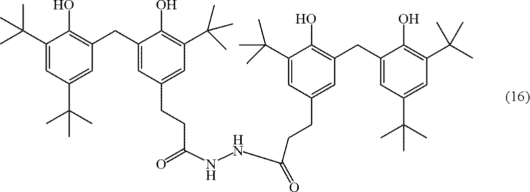 [SMILES: CC(C)(C)c1cc(Cc2cc(CCC(=O)NNC(=O)CCc3cc(Cc4cc(C(C)(C)C)cc(C(C)(C)C)c4O)c(O)c(C(C)(C)C)c3)cc(C(C)(C)C)c2O)c(O)c(C(C)(C)C)c1]

The procedure of Example 12 was followed, but hydrazine hydrate was used in place of hexamethylenediamine. Compound (16) is obtained. MS(m/z) = 876.6.

### Example 17

### Pentaerythritol tetrakis(3-(3-(tert-butyl)-5-(3,5-di-tert-butyl-2-hydroxybenzyl)-4-hydroxyphenyl)phen ylpropionate

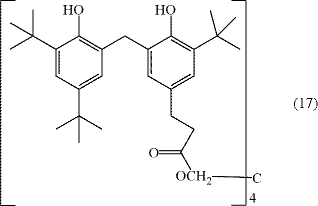

According to the method according to Example 10, but using compound (1) and pentaerythritol. Compound (17) is obtained. MS(m/z) = 1945.2.

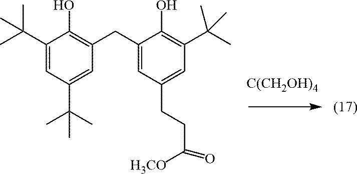

### Example 18

### N,N″,N″-(1,3,5-triazine-2,4,6-triyl)tris(3-(3-(tert-butyl)-5-(3,5-di-tert-butyl) Butyl-2-hydroxybenzylbenzyl)-4-hydroxyphenyl)propionamide)

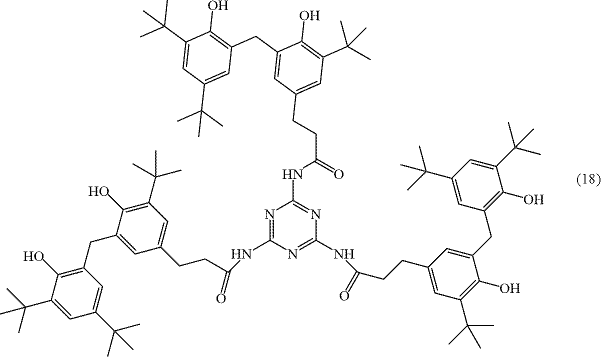 [SMILES: CC(C)(C)c1cc(Cc2cc(CCC(=O)Nc3nc(NC(=O)CCc4cc(Cc5cc(C(C)(C)C)cc(C(C)(C)C)c5O)c(O)c(C(C)(C)C)c4)nc(NC(=O)CCc4cc(Cc5cc(C(C)(C)C)cc(C(C)(C)C)c5O)c(O)c(C(C)(C)C)c4)n3)cc(C(C)(C)C)c2O)c(O)c(C(C)(C)C)c1]

The procedure of Example 12 was followed, but melamine was used instead of hexamethylenediamine. 150 g of 3-(3-(tert-butyl)-5-(3,5-di-tert-butyl-2-hydroxybenzyl)-4-hydroxyphenyl)propionyl chloride and 12.6 g of melamine were used. Compound (18) is obtained. MS(m/z) = 1392.9.

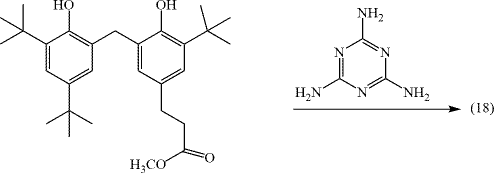

### Example 19

### (2,4,6-Trioxo-1,3,5-triazine-1,3,5-triyl)tris(ethane-2,1-diyl)tris(3-(3-(tert. butyl)-5-(3,5-di-tert-butyl-2-hydroxybenzyl)-4-hydroxyphenyl)proplonate)

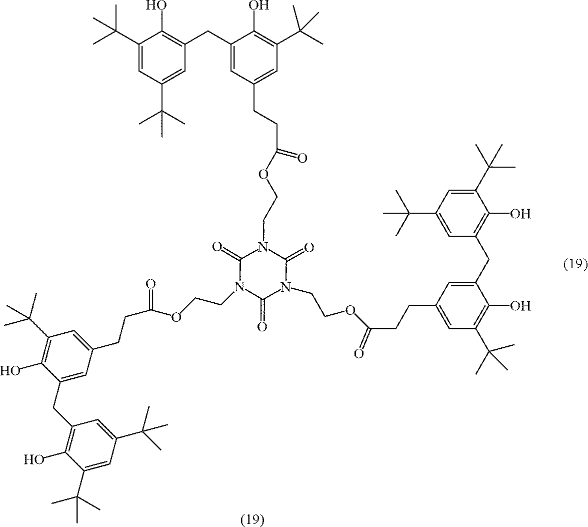

The method of Example 10 was followed, but using 150 g of compound (1) and 26 g of trishydroxyethyl isocyanurate. Trihydroxyethyl isocyanurate is an industrial raw material (839-90-7, mp=136° C.). Compound (19) is obtained. MS(m/z)= 1527.9.

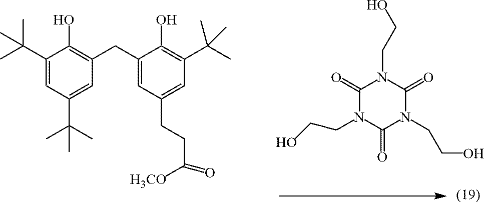

### Example 20

### (2,4,6-Trimethylbenzene-1,3,5-triyl)tris(methylene)tris(3-(3-(tert-butyl)-5-(3,5-di-tert-butyl) yl-2-hydroxybenzyl)-4-hydroxyphenyl)proplonate)

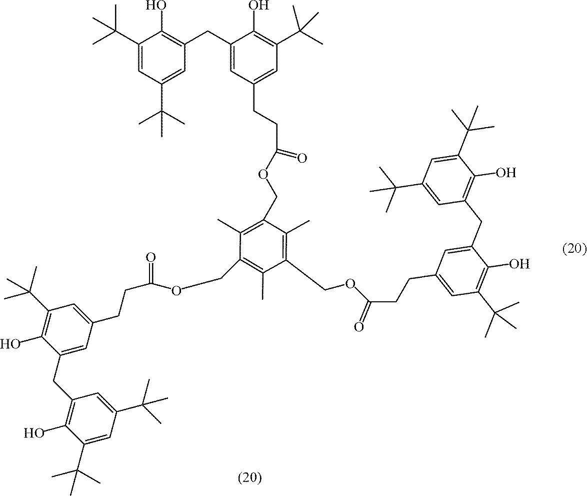

The procedure of Example 10 was followed, but using 150 g of compound (1) and 21 g of 2,4,6-trimethylbenzene-1,3,5-triyl)trimethanol. Compound (20) isobtained. MS(m/z) = 1477.0.

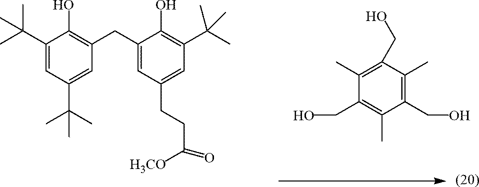

### Example 21

### (Mono-hexaylbenzene)tris(methylene)tris(3-(3-(tert-butyl)-5-(3,5-di-tert-butyl-2-hydr oxybenzyl)-4-hydroxyphenyl) ) propionate) mixture

According to the method of Example 10, but using 30 g of compound (1) and 2.6 g of 1,2,3,4,5,6 hexamethanolbenzene. The mixture (21) is obtained.

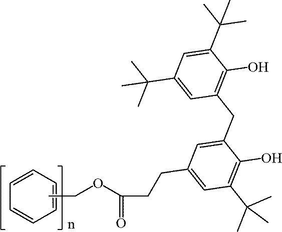

### Example 22

### 1,2,3-Tris(3-(3-(tert-butyl)-5-(3,5-di-tert-butyl-2-hydroxybenzyl)-4-hydroxyphenyl)pr opionate phenyl ester)propane

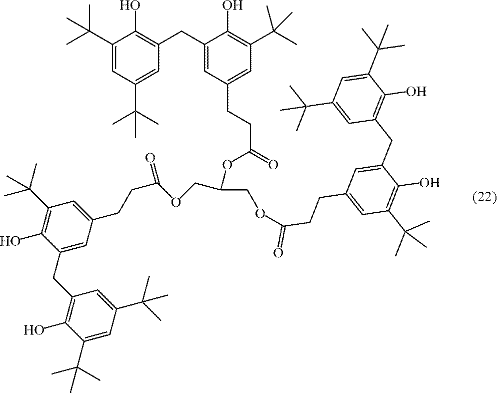 [SMILES: CC(C)(C)c1cc(Cc2cc(CCC(=O)OCC(COC(=O)CCc3cc(Cc4cc(C(C)(C)C)cc(C(C)(C)C)c4O)c(O)c(C(C)(C)C)c3)OC(=O)CCc3cc(Cc4cc(C(C)(C)C)cc(C(C)(C)C)c4O)c(O)c(C(C)(C)C)c3)cc(C(C)(C)C)c2O)c(O)c(C(C)(C)C)c1]

According to the method of Example 10, but using 150 g of compound (1) and 9.2 g of glycerol. Compound (22) is obtained. MS(m/z) = 1358.9.

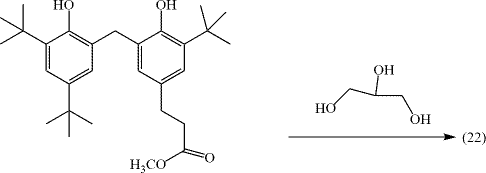

### Example 23

### Calcium 3-(3-(tert-butyl)-5-(3,5-di-tert-butyl-2-hydroxybenzyl)-4-hydroxyphenyl)propionate

45.4 g of compound (1) was stirred in 200 ml of alcohols mixed solution under nitrogen. About 100 ml of 5% NaOH solution was added dropwise. After the dropwise addition was completed, it was slowly heated to 60° C. and reacted for 4 hours. The alcohol solvent was removed by rotary evaporation, and 100 ml of ethyl acetate was added for extraction. The aqueous layer was taken, and diluted hydrochloric acid was added dropwise to neutralize to pH=7-8, and 0.5 M calcium dichloride aqueous solution was gradually added at the same time, stirred for 2 hours, allowed to stand, and filtered. The free acid was removed by washing with potassium carbonate aqueous solution, washed with water until neutral, and dried to obtain compound (23)

### Example 24

### 3-(5-(Tert-butyl)-2-hydroxy-3-(2-phenylpropan-2-yl)benzyl)-4-hydroxy-5-(2-pheny lpropane-2- yl) phenylmethyl) methyl propionate

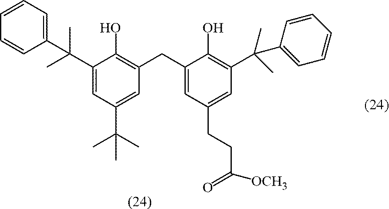

According to the method of Example 1, but using 4-(tert-butyl)-2-(2-phenylpropan-2-yl)phenol (compound 24-1) and 3-(4-hydroxy-3-(2-benzene) The methyl propan-2-yl)phenyl)propionate compound (24-2) was reacted. Compound (24) is obtained.

The preparation method of compound (24-1) or compound (24-2) is as follows: According to the method of Example 1, 15 g of 4-tert-butylphenol (or 29.8 g of 3-(4-hydroxy-3-(2-phenyl) Propan-2-yl)phenyl)propionic acid methyl ester) and 15.4 g of 2-chloro-2-phenylpropane (CAS RN, 515-40-2), add 200 mL of dichloromethane, under nitrogen stirring, add anhydrous 14 g AlCl₃ and stir overnight. TLC monitoring showed that the reaction was complete. The reaction mixture was poured into 200 mL of ice water, stirred, and extracted three times with CH₂Cl₂. The extract phases were combined, washed successively with 1% dilute hydrochloric acid and brine, and dried over anhydrous sodium sulfate. The solvent was evaporated to dryness on an evaporator, and the obtained residue was purified by column chromatography to obtain compound (24-1), MS (m/z) = 268.2 or compound (24-2). MS(m/z) = 298.2.

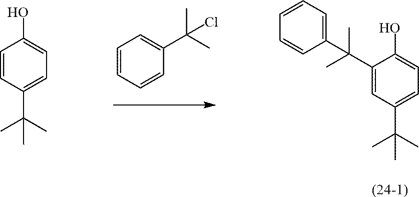

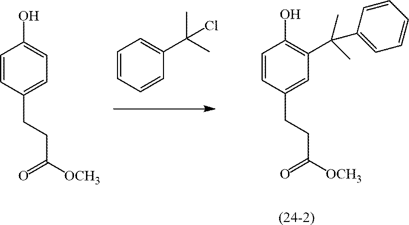

### Example 25

### Methyl 3-(3-(5-(tert-butyl)-2-hydroxybenzyl)-5-((dodecylthio)methyl)-4-hydroxyphenyl)prop anoate

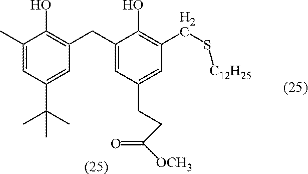

According to the method of Example 1, but using methyl 3-(3-(dodecylthio)-4-hydroxyphenyl)propanoate in place of compound (1), compound (25) was obtained. MS(m/z) = 570.4.

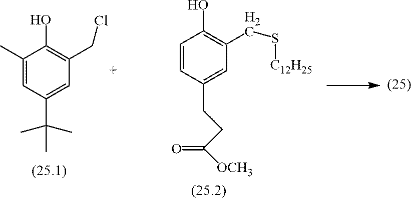

3-(3-(dodecylthio)-4-hydroxyphenyl) methyl propionate production method: 100 g of 4-hydroxyphenyl-propionate methyl ester is added to 86 g of dodecanethiol, paraformaldehyde 19 g, 150 mL of dimethylformamide, and 3.6 g of piperidine and were heated to reflux ovemight under nitrogen protection. Compound (25.2) was obtained after filtration, washing with water and suction filtration.

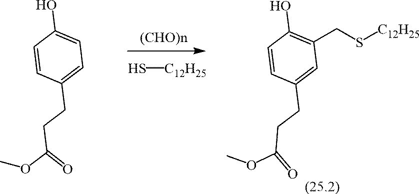

### Example 26

### Methyl 3-(3-(tert-butyl)-5-(3,5-di-tert-butyl-4-hydroxybenzyl)-4-hydroxyphenyl)propanoate

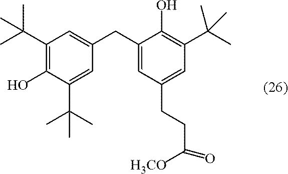 [SMILES: COC(=O)CCc1cc(Cc2cc(C(C)(C)C)c(O)c(C(C)(C)C)c2)c(O)c(C(C)(C)C)c1]

Take 25.4 g of 2,6-di-tert-butyl-4-chloromethylphenol (CAS No 955-01-1, mp=40° C.) and 29 g of 3-(3-(tert-butyl)-4-hydroxybenzene base) methyl propionate (CAS No 36837-50-0, mp=60° C.), dissolved in 200 mL of dry CH2Cl2, stirred at room temperature under nitrogen, and stirred with anhydrous 14 g of AlCl3. The reaction was monitored by TLC during which AlCl3 was supplemented. After the completion of the reaction, the reaction mixture was poured into 200 mL of ice water, stirred, and extracted with CH₂Cl₂ three times. The extract phases were combined, washed successively with 1% dilute hydrochloric acid and brine, and dried over anhydrous sodium sulfate. The solvent was evaporated to dryness on an evaporator, and the obtained residue was purified by column chromatography to obtain compound (26). MS(m/z) = 454.3.

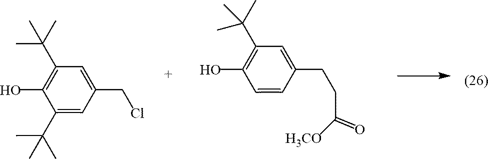

### Example 27

### Methyl 3-(3-(tert-butyl)-5-(3,5-di-tert-butyl-4-hydroxybenzoyl)-4-hydroxyphenyl)propanoate

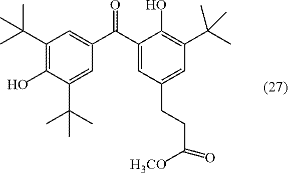 [SMILES: COC(=O)CCc1cc(C(=O)c2cc(C(C)(C)C)c(O)c(C(C)(C)C)c2)c(O)c(C(C)(C)C)c1]

Following the procedure of Example 26, but substituting 3,5-di-tert-butyl-4-hydroxybenzoyl chloride for 2,6-di-tert-butyl-4-chloromethylphenol, compound (27) was obtained. MS(m/z) = 468.3.

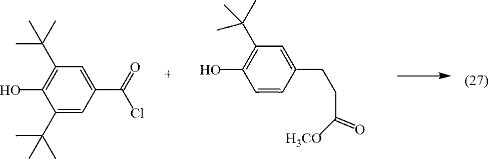

### Example 28

### Methyl 3-(3-tert-butyl-5-((3,5-di-tert-butyl-4-hydroxyphenyl)(hydroxy)methyl)-4-hydroxyphe nyl)propanoate

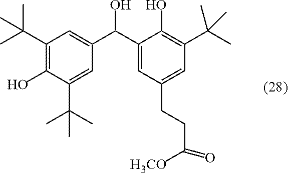 [SMILES: COC(=O)CCc1cc(C(O)c2cc(C(C)(C)C)c(O)c(C(C)(C)C)c2)c(O)c(C(C)(C)C)c1]

Following the procedure of Example 3, but substituting compound (27) for compound (2), compound (28) was obtained. MS(m/z) = 470.3.

### Example 29

### Methyl 3-(3-(tert-butyl)-5-(1-(3,5-di-tert-butyl-4-hydroxyphenyl)ethyl)-4-hydroxyphenyl)pro panoate

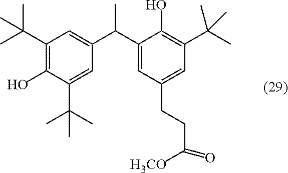 [SMILES: COC(=O)CCc1cc(C(C)c2cc(C(C)(C)C)c(O)c(C(C)(C)C)c2)c(O)c(C(C)(C)C)c1]

Following the procedure of Example 1, but substituting acetaldehyde for polyoxymethylene, compound (29) was obtained. MS(m/z) = 468.3.

### Example 30

### Methyl 3-(3-(tert-butyl)-5-(3-(tert-butyl)-4-hydroxy-5-methylbenzyl)-4-hydroxyphenyl)propa noate

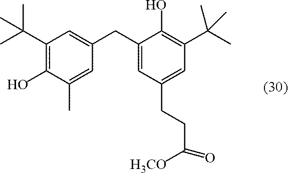 [SMILES: COC(=O)CCc1cc(Cc2cc(C)c(O)c(C(C)(C)C)c2)c(O)c(C(C)(C)C)c1]

According to the method of Example 26, but using 2-(chloromethyl)-4-(tert-butyl)-6-methylphenol instead of 2,4-di-tert-butyl-6-chloromethylphenol, compound ( 30) was obtained. MS(m/z) = 412.3.

### Example 31

### Octyl 3-(3-(tert-butyl)-5-(3,5-di-tert-butyl-4-hydroxybenzyl)-4-hydroxyphenyl)propanoate

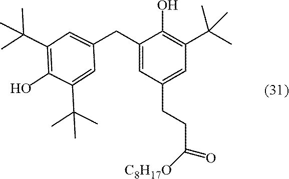

According to the method of Example 6, except that compound (26) was used in place of compound (1), compound (31) was obtained. MS(m/z) = 552.4.

### Example 32

### 3-(3,5-Di-tert-butyl-5-(3,5-di-tert-butyl-4-hydroxybenzyl)-4-hydroxyphenyl)propionic acid octadecyl ester

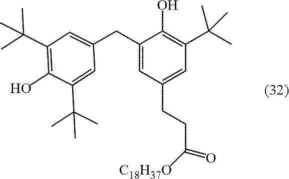

According to the method of Example 7, except that compound (26) is used in place of compound (1), compound (32) is obtained. MS(m/z) = 692.6.

### Example 33

### Oxobis(ethane-2,1-diyl)bis(3-(3-(tert-butyl)-5-(3,5-di-tert-butyl-4-hydroxybenzyl)-4-h ydroxybenzene base) propionate)

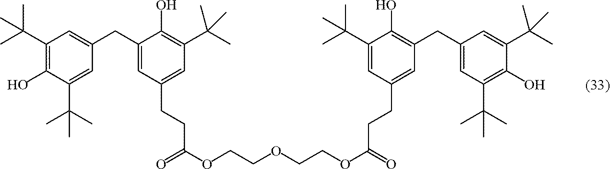 [SMILES: CC(C)(C)c1cc(CCC(=O)OCCOCCOC(=O)CCc2cc(Cc3cc(C(C)(C)C)c(O)c(C(C)(C)C)c3)c(O)c(C(C)(C)C)c2)cc(Cc2cc(C(C)(C)C)c(O)c(C(C)(C)C)c2)c1O]

120 g of compound (26), 13 g of diethylene glycol, 0.5 g of aluminum triisopropoxide (in toluene) were mixed. The reaction mixture was stirred and heated to 85° C. under nitrogen atmosphere, and the resulting methanol was condensed and removed by vacuum. The reaction was monitored, and when the reaction was complete, aqueous citric acid (50%) was added and stirring continued for 20 minutes. Then, water was added and stirred for 20 minutes at 75° C. The organic phase was separated and washed twice with brine, then dried over sodium sulfate. Then toluene and excess octanol were distilled off from the organic phase under reduced pressure, and the residue was dried in vacuo. Chromatography gave compound (33). MS(m/z) = 950.6.

### Example 34

### (Ethan-1,2-diylbis(oxy))bis(ethane-2,1-diyl)bis(3-(3-(tert-butyl)-5-(3,5-di-tert-butyl) Butyl-4-)hydroxybenzyl)-4-hydroxyphenyl)propionate)

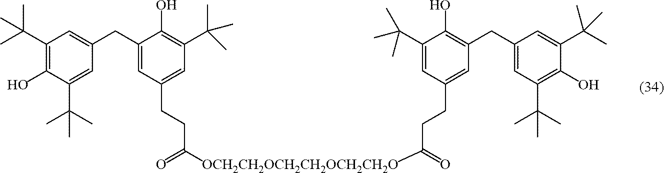

Following the procedure of Example 33, but substituting triethylene glycol for diethylene glycol, compound (34) was obtained. MS(m/z) = 994.7.

### Example 35

### Thiobis(ethane-2,1-diyl)bis(3-(3-(tert-butyl)-5-(3,5-di-tert-butyl-4-hydroxybenzyl)-4-hydroxybenzene base) propionate)

Following the procedure of Example 33, but substituting 2,2′-thiodiethanol for diethylene glycol, compound (35) was obtained. MS(m/z) =966.6

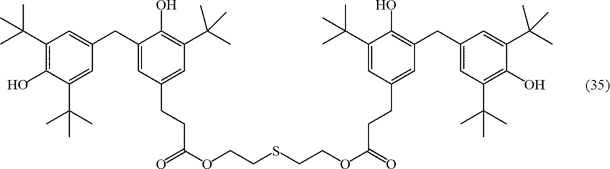 [SMILES: CC(C)(C)c1cc(CCC(=O)OCCSCCOC(=O)CCc2cc(Cc3cc(C(C)(C)C)c(O)c(C(C)(C)C)c3)c(O)c(C(C)(C)C)c2)cc(Cc2cc(C(C)(C)C)c(O)c(C(C)(C)C)c2)c1O]

### Example 36

### 3,6,9,12,15,18,21,24-Octaoxohexadecane-1,26-diylbis(3-(3-(tert-butyl)-5-(3,5-Di-tert-butyl-4-)hydroxybenzyl)-4-hydroxyphenyl)propionate)

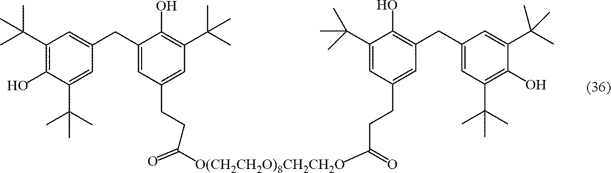

According to the method of Example 33, but substituting nonaethylene glycol for diethylene glycol, compound (36) was obtained by chromatography.

### Example 37

### N,N′-(Propane-1,3-diyl)bis(3-(3-tert-butyl-5-(3,5-di-tert-butyl-4-hydroxybenzyl)-4-hy droxyphenyl)) propionamide)

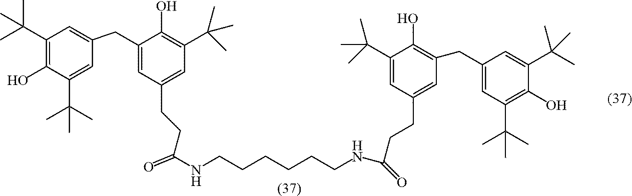

According to the method of Example 12, but replacing compound (1) with compound (26), compound (37) was obtained. MS(m/z) = 960.7.

### Example 38

### 1,6-Diylbis(3-(3-tert-butyl-5-(3,5-di-tert-butyl-4-hydroxybenzyl)-4-hydroxyphenyl)pr opionate hexyl)

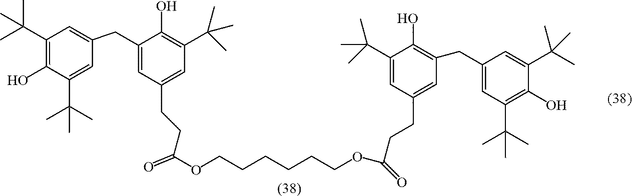

Following the procedure of Example 13, but substituting compound (26) for compound (1), compound (38) was obtained. MS(m/z) = 962.7.

### Example 39

### Bis(ethane-2,1-diyl)bis(3-(3-tert-butyl-5-(3,5-di-tert-butyl-4-hydroxybenzyl))-4-hydro xyphenyl)propane Acid (oxalyl bis(aza-diyl)) ester

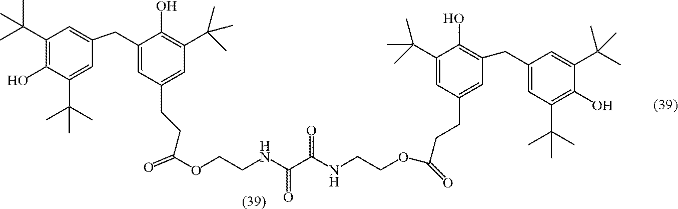

According to the method according to Example 14, but using compound (26) instead of compound (1). Compound (39) is obtained. MS(m/z) = 1020.6.

### Example 40

### (2,4,8,10-Tetraoxaspiro[5.5]undecan-3,9-diyl)bis(2-methylpropane-2,1-diyl)bis(3-(3-t ert.) butyl)-5-(3,5-di-tert-butyl-4-hydroxybenzyl)-4-hydroxyphenyl)propionate)

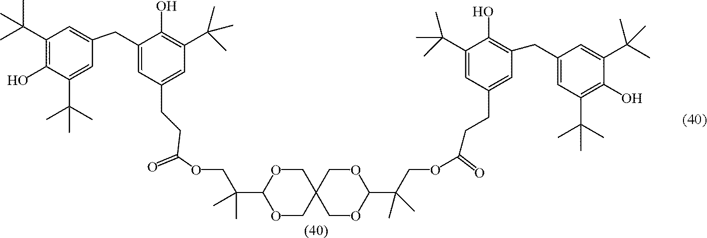

The procedure of Example 15 was followed, but compound (26) was used in place of compound (1). Compound (40) is obtained. MS(m/z) = 1148.8.

### Example 41

### N(3-tert-butyl-5-(3,5-di-tert-butyl-4-hydroxybenzyl)-4-hydroxyphenyl)-N′-(3-(3-te rt-butyl-5-(3,5-Di-tert-butyl-4-hydroxybenzyl)-4-hydroxyphenyl)propionyl)propanehydrazide

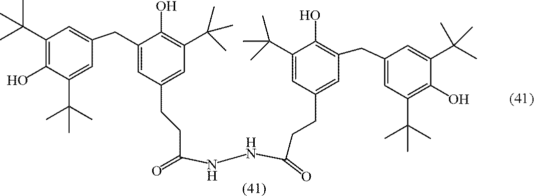

The procedure of Example 16 was followed, but compound (26) was used in place of compound (1). Compound (41) is obtained. MS(m/z) = 876.6.

### Example 42

### Pentaerythritol tetrakis(3-(3-tert-butyl)-5-(3,5-di-tert-butyl-2-hydroxybenzyl)-4-hydroxyphenyl)phen ylpropionate

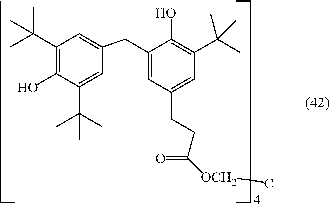

The procedure of Example 17 was followed, but compound (26) was used in place of compound (1). Compound (42) is obtained. MS(m/z) = 1945.2.

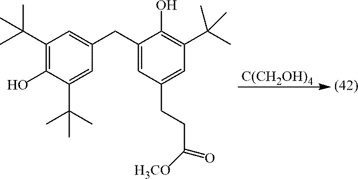

### Example 43

### N,N″,N‴-(1,3,5-triazine-2,4,6-triyl)tris(3-(3-(tert-butyl)-5-(3,5-Di-tert-butyl-4-hydroxybenzyl)-4-hydroxyphenyl)propionamide)

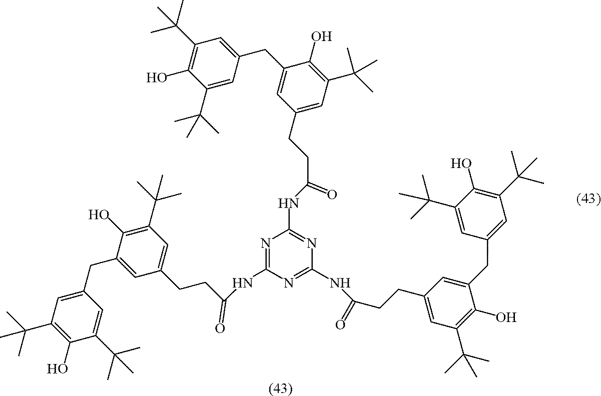

The procedure of Example 18 was followed, but compound (26) was used in place of compound (1). Compound (43) is obtained. MS(m/z) = 1392.9.

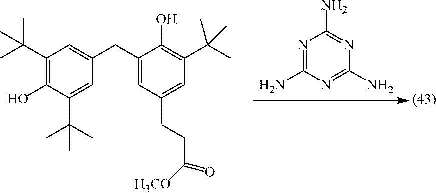

### Example 44

### (2,4,6-Trioxo-1,3,5-triazine-1,3,5-triyl)tris(ethane-2,1-diyl)tris(3-(3-(tert. butyl)-5-(3,5-di-tert-butyl-4-hydroxybenzyl)-4-hydroxyphenyl)propionate)

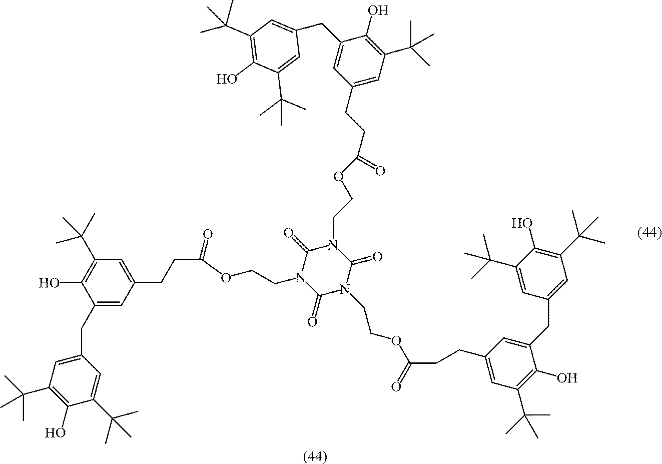

According to the method of Example 19, but using compound (26) instead of compound (1). Compound (44) is obtained. MS(m/z) = 1527.9.

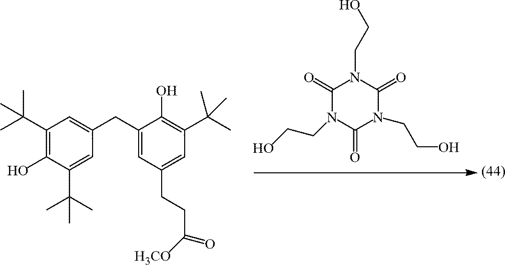

### Example 45

### 1,3,5-Triyltris(ethane-2,1-diyl)tris(3-(3-(tert-butyl)-5-((3,5-di-tert-butyl-4-hydroxyben zyl) base)-4-hydroxyphenyl)phenyl propionate)

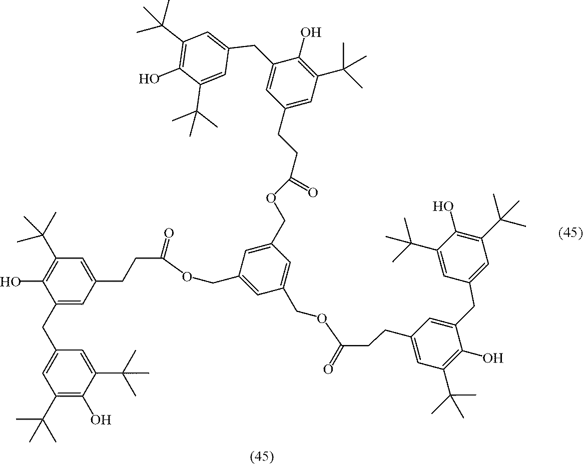

The procedure of Example 20 was followed, but compound (26) was used in place of compound (1). 1,3,5 Benzenetrimethanol (4464-18-0) was used in place of 2,4,6-trimethylbenzene-1,3,5-triyl)trimethanol. Compound (45) is obtained. MS (m/z) 1434.9.

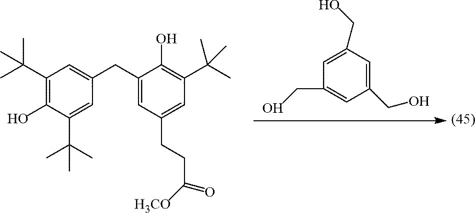

### Example 46

### 1,2,3-Tris(3-(3-(tert-butyl)-5-(2,4-di-tert-butyl-4-hydroxybenzyl)-4-hydroxyphenyl)pr opionate phenyl ester)propane

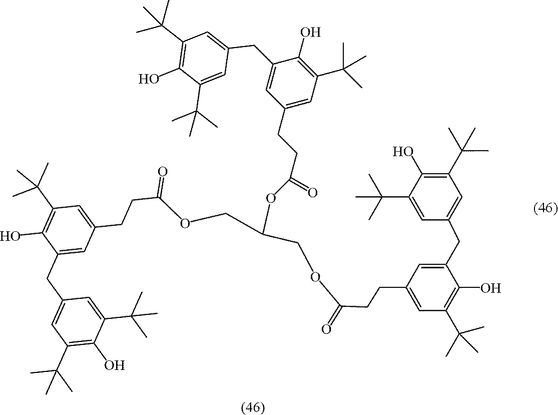

The procedure of Example 22 was followed, but compound (26) was used in place of compound (1). Compound (45) is obtained. MS(m/z) = 1358.9.

### Example 47

### 3-(Tert-butyl)-4-hydroxy-5-(2-phenylpropan-2-yl)benzyl)-4-hydroxy-5-(2-pheny lpropane-2- yl) phenylmethyl) methyl propionate

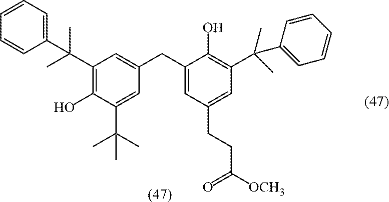

The procedure of Example 24 was followed, but using 2-(tert-butyl)-4-(chloromethyl)-6-(2-phenylpropan-2-yl)phenol (compound 47.1) instead of compound (24.1). Compound (47) is obtained. MS(m/z)=578.3.

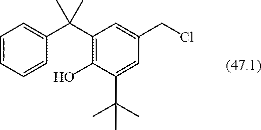 [SMILES: CC(C)(C)c1cc(CCl)cc(C(C)(C)c2ccccc2)c1O]

### Example 48

### Hexane-1,6-diylbis(3-(3-(3-(tert-butyl)-4-hydroxy-5-(2-phenylpropan-2-yl)benzyl)-4-hydroxy- 5-(2-Phenylpropan-2-yl)phenyl)propionic acid methyl ester)

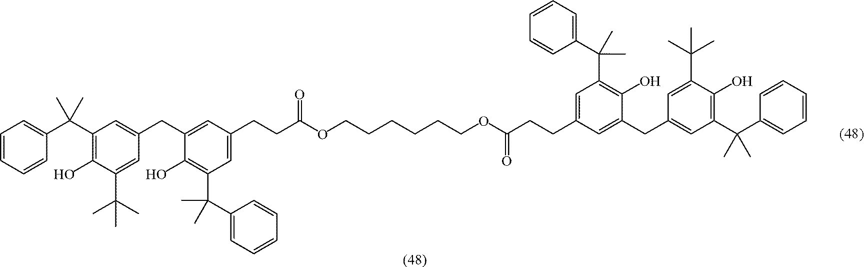

According to the method of Example 13, but using compound (47) instead of compound (1). Compound (48) is obtained. MS(m/z)=1210.7.

### Example 49

### Methyl 3-(3-(3,5-di-tert-butyl-4-hydroxybenzyl)-5-((dodecylthio)methyl)-4-hydroxyphenyl)pr opanoate

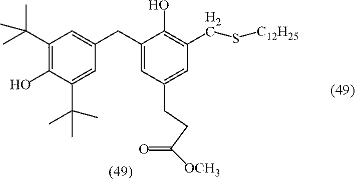

According to the method of Example 25, but using 2,6-di-tert-butyl-4-(chloromethyl)phenol instead of compound (25.1). Compound (49) is obtained. MS(m/z) = 612.4.

### Example 50

### Butane-1,4-diylbis(3-(3-(3,5-di-tert-butyl-4-hydroxybenzyl)-5-((dodecylthio)methyl)-4- hydroxyphenyl) propionate)

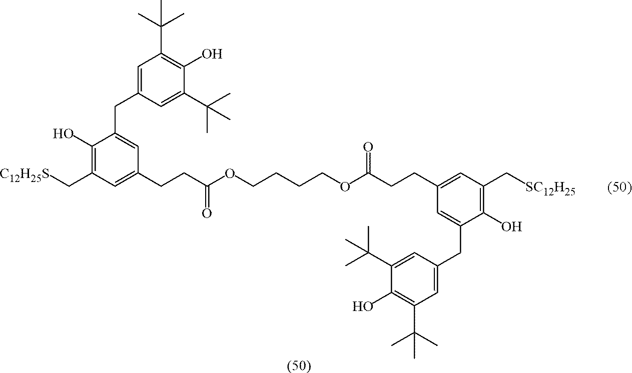

The procedure of Example 13 was followed, but instead of compound (1), substitute compound (49) was used. Butanediol replaces hexanediol. Compound (50) is obtained. MS(m/z) = 1250.9.

### Example 51

### Methyl 3-(3,5-di-tert-butyl-2-(3,5-di-tert-butyl-4-hydroxybenzyl)-4-hydroxyphenyl)propanoat e

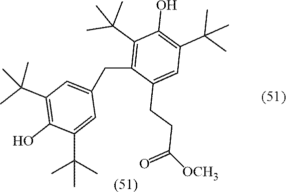

25.4 g of 3,5-di-tert-butyl-4-hydroxybenzyl chloride (CAS No 955-01-1, mp=40° C.) and 29.2 g of (3,5-di-tert-butyl-4-hydroxybenzene base) methyl propionate (CAS No 6386-38-5, mp=66° C.), dissolved in 200 mL of dry CH2Cl2, added anhydrous 14 g of AlCl3 and stirred at room temperature under nitrogen. The reaction was monitored by TLC during which AlCl3 was supplemented. After the completion of the reaction, the reaction mixture was poured into 200 mL of ice water, stirred, and extracted with CH₂Cl₂ three times. The extract phases were combined, washed successively with 1% dilute hydrochloric acid and brine, and dried over anhydrous sodium sulfate. The solvent was evaporated to dryness, and the obtained residue was purified using column chromatography to obtain compound (51). MS(m/z) = 510.4.

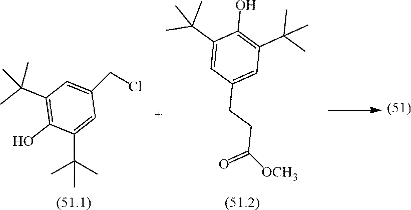

### Example 52

### Methyl 3-(3,5-di-tert-butyl-2-(3,5-di-tert-butyl-4-hydroxybenzoyl)-4-hydroxyphenyl)propano ate

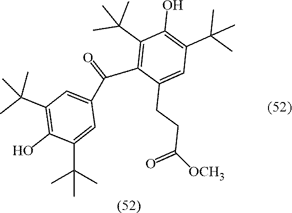

Following the procedure of Example 51, but substituting 3,5-di-tert-butyl-4-hydroxybenzoyl chloride for 3,5-di-tert-butyl-4-hydroxybenzyl chloride, compound (2) was obtained. MS(m/z) = 524.4.

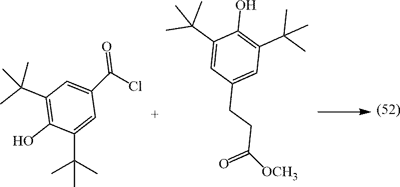

### Example 53

### Methyl 3-(3,5-di-tert-butyl-2-((3,5-di-tert-butyl-4-hydroxyphenyl)(hydroxy)methyl)-4-hydrox yphenyl)propanoate

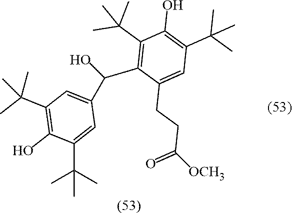

Following the procedure of Example 3, but substituting compound (52) for compound (2), compound (53) was obtained. MS(m/z) = 526.4.

### Example 54

### Methyl 3-(3,5-di-tert-butyl-2-(chloro(3,5-di-tert-butyl-4-hydroxyphenyl)methyl)-4-hydroxyph enyl)propanoate

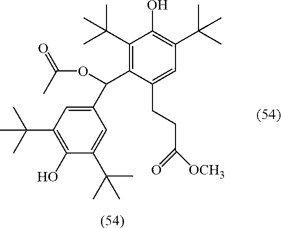

Dissolve 51 g of compound (53) in toluene, add 6 g of acetic acid and 1.5 mL of concentrated sulfuric acid under the configuration of a condensing water separator, heat to reflux at 110° C., and monitor the reaction. After the reaction is completed, add saturated NaHCO3 for neutralization, and then extracted with dichloromethane. It was then washed with saturated NaCl and dried over anhydrous MgSO4. After filtration, it was evaporated to dryness and passed through columnchromatography to obtain compound (54). MS(m/z) = 568.4.

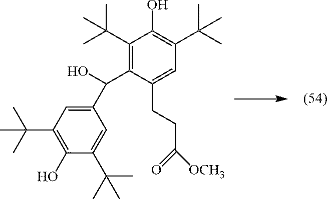

### Example 55

### Methyl 3-(5-(tert-butyl)-2-(1-(3,5-di-tert-butyl-2-hydroxyphenyl)-2-phenethyl)-4-hydroxyphe nyl)propanoate

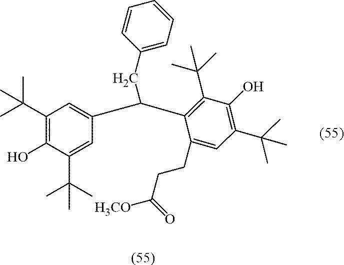

According to the method of Example 4, but with phenylacetaldehyde instead of acetaldehyde, compound 2,6-di-tert-butylphenol instead of compound (4.1), compound (51.2) instead of compound (1.2). Chromatography gave compound (55). MS(m/z) = 600.4.

### Example 56

### Octyl 3-(3,5-Di-tert-butyl-2-(3,5-di-tert-butyl-4-hydroxybenzyl)-4-hydroxyphenyl)propanoa te

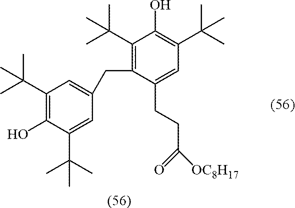

Following the procedure of Example 6, but substituting compound (51) for compound (1), compound (56) was obtained. MS(m/z) = 608.5.

### Example 57

### (Ethan-1,2-diylbis(oxy))bis(ethane-2,1-diyl)bis(3-(5-(tert-butyl)-2-(3,5-di-tert-butyl) yl-4-)hydroxybenzyl)-4-hydroxyphenyl)propionate)

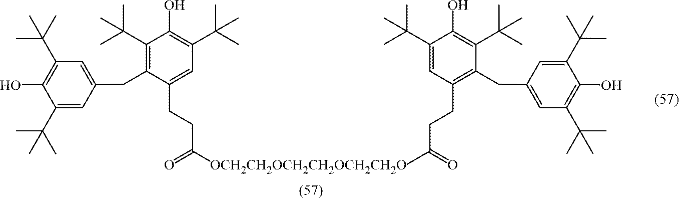

Following the procedure of Example 10, but substituting compound (51) for compound (1), compound (57) was obtained. MS(m/z) = 1106.8.

### Example 58

### 3-(tert-butyl)-2-(3,5-di-tert-butyl-4-hydroxybenzyl)-4-hydroxyphenyl)-N′-(3-(te rt-butyl))-2--(3,5-di-tert-butyl-4-hydroxybenzyl)-4-hydroxyphenyl)propionyl)propan ehydrazide

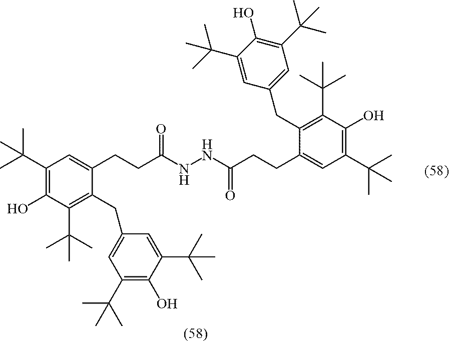

Following the procedure of Example 16, but substituting compound (51) for compound (1), compound (58) was obtained. MS(m/z) = 988.7.

### Example 59

### 2-(Acetoxyimino)-1-(4-((4-(3-oxo-3-phenylprop-1-en-1-yl)phenyl)sulfanyl)phenyl)he x-1-keto

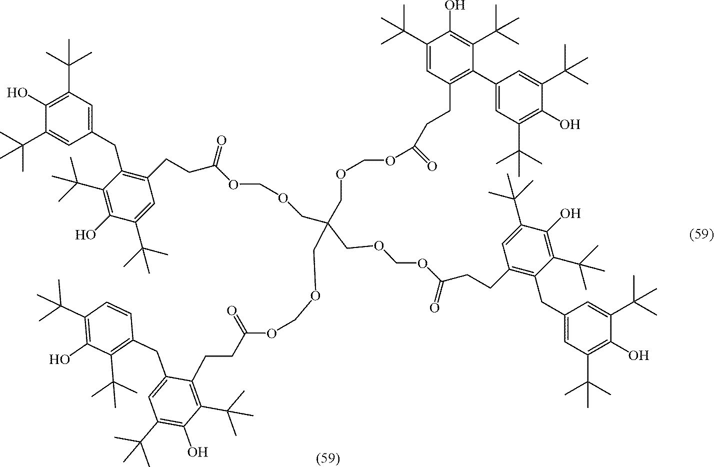

Following the procedure of Example 17, but substituting compound (51) for compound (1), compound (59) was obtained. MS(m/z) = 2155.5.

### Example 60: Antioxidant Extraction Test in Polyurethane

Polyether polyols are polyurethane raw materials. After mixing 50 parts by weight of polyether polyol (triol, molecular weight 3000), 2.0 parts by weight of water, 0.1 parts by weight of triethylenediamine, and 1.0 parts by weight of silicone oil. A mixture comprising 0.2 parts by weight of stannous octoate, 0.15 parts by weight of example compounds or control compounds, 50 parts by weight of toluene diisocyanate, 50 parts by weight of polyether polyol (triol, molecular weight 3000) was added. After the two are mixed, they are poured into a box for foaming reaction. Let stand for 1 hour at room temperature and mature in an oven. After the reaction was complete, a 1-gram sample of polyurethane with or without antioxidants was cut and placed in a glass jar with a lid for anti-extraction or anti-aging analysis. Add 100ml of solvent for extraction, and analyze the extract. Extraction of various compounds was detected by HPLC. The amount extracted from the control group was 100%. The smaller the amount extracted, the less likely it is to separate out. Basically, the anti-aging or anti-yellowing ability of the tested compounds is positively correlated with the number of hindered phenolic units. The results of the anti-extraction test are shown in Table 4. Taking the control group to be extracted as 100%, the relative ratio of the extracted compounds of the example compounds was converted. The extracted percentage of the example compound=(100÷the extracted amount of the control group a)×(the extracted amount of the example compound b)×%. The overall antioxidant efficiency of the example compounds is proportional to the residual amount in the polyurethane after extraction and is proportional to (number of hindered phenolic units/molecular weight). For example, the ratio of (hindered phenol unit/molecular weight) of compound 10 and Eunox 1035 is (4/966): (2/642)=1.33, as long as the residual ratio is greater than 1, it can be presumed to be progressive. The extracted percentages of the compounds of the examples in Table 4 are all less than 75% to the controls, that is, the ratios are all greater than 1.

TABLE 4

The extraction test for antioxidants

| Example compounds | 1-5, 24-30 47,49 51-55 | 6, 31, 56 | 7, 32 | 8 | 9 | 10, 35 | 11, 33,34, 36,57 | 12, 37 | 13, 38,48, 50 |
|---|---|---|---|---|---|---|---|---|---|
| Control compounds (the extracted ratio as 100%) | | | | | | | | | |
| AO 35 | 70% below | - | - | - | - | - | - | - | - |
| Eunox 1135 | | 70% below | | | | | | | |
| Eunox 1076 | | | 70% below | | | | | | |
| 41028-42-6 | | | | 75% below | | | | | |
| 41028-42-6 | | | | | 70% below | | | | |
| Eunox 1035 | | | | | | 75% below | | | |
| Eunox 245 | | | | | | | 75% below | | |
| Eunox 1098 | | | | | | | | 75% below | |
| AO 259 | | | | | | | | | 75% below |

TABLE 4

continmtion

| Example compounds | 14, 39 | 15, 40 | 16, 41,58 | 17, 42,59 | 18-19, 43-44 | |
|---|---|---|---|---|---|---|
| Control compounds (the extracted ratio as 100%) | | | | | | |
| MD 697 | 75% below | | | | | |
| AO 80 | | 75% below | | | | |
| Eunox 1024 | | | 75% below | | | |
| Eunox 10 | | | | 75% below | | |
| Eunox 3114 | | | | | 75% below | |
| Eunox 1330 | | | | | | 75% below |

The reference or control substances are all from commercial products or patents, Eunox is the trademark of the applicant, 41028-42-6 (CAS no) is from the patent (JP56052073), and the structural formula is as follows:

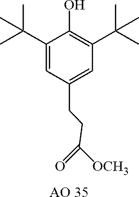

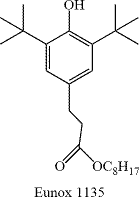

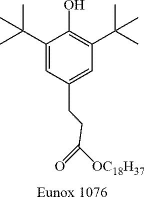

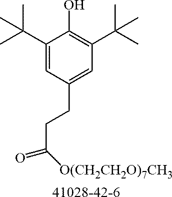

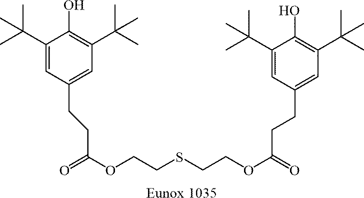

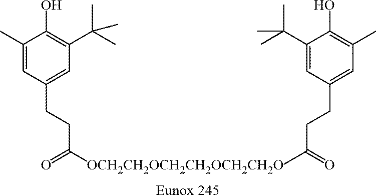

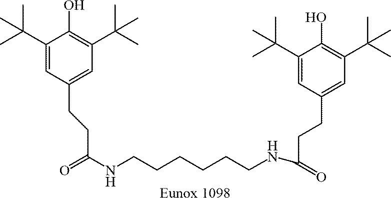

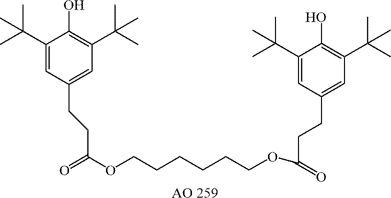

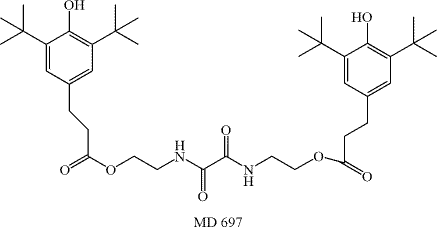

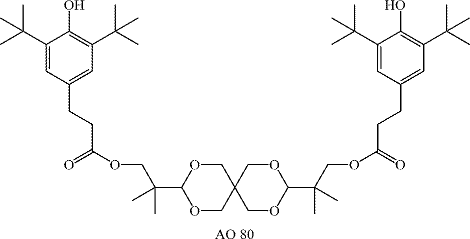

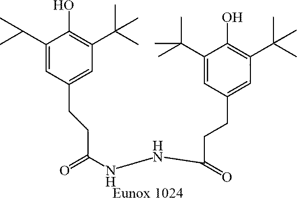

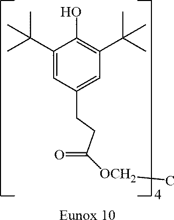

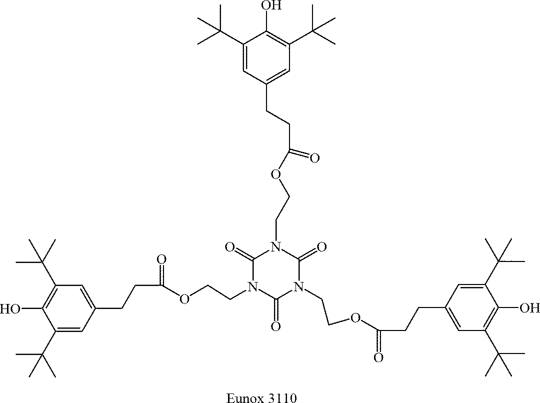

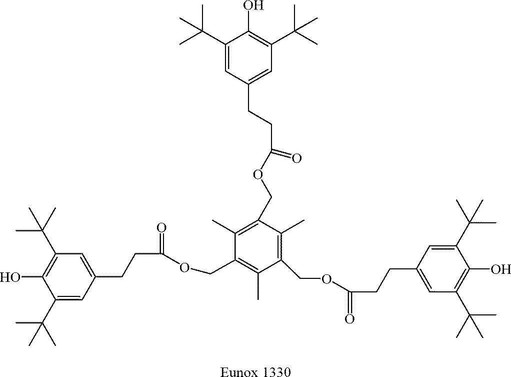

The present invention has been disclosed above with preferred embodiments, but it is not intended to limit the present invention, and all technical solutions obtained by adopting equivalent replacement or equivalent transformation methods fall within the protection scope of the present invention.

What is claimed is:

1. A compound of formula (I) or a salt thereof, 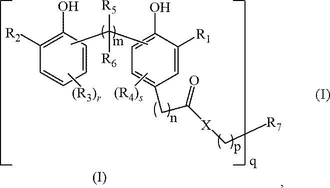
wherein, R₁, R₂ are each independently selected from C₁~C₁₀-containing alkyl, phenyl, benzyl, cumyl, C₁~C₁₂ sulfane or C₁~C₂ methylene C₁~C₁₂ sulfane; R3, R4 is independently selected from hydrogen, C1~C6 alkyl, phenyl, benzyl, cumyl, C₁~C₁₂ sulfane or C₁~C₂ methylene C₁-C₁₂ sulfane; R₅, R₆ are independently selected from hydrogen, hydroxyl, halogen, carbonyl, carboxyl, acyl, ester group, phenyl, C₁~C₆ alkyl, C₁~C₆ alkylamino, C₁~C₆ alkoxy, or R₅~R₆ are combined into a ketone group;
R₇ is a q-valent group;
X is selected from N, NH, NHR₈, O, S, CH₂, CHR₈, R₈ is selected from H, OH, C1-C6 alkyl; m=0~3; n=0~3; p=0~18; q=1~8; r=0~3; s=0~2.

2. The compound of claim 1, wherein R₇ is selected from a bond, hydrogen, unsubstituted or substituted carbon or carbon chain, unsubstituted or substituted oxygen or sulfur or nitrogen or metal atom, unsubstituted or substituted carbon chains interrupted by oxygen or sulfur or nitrogen, unsubstituted or substituted 5-7 membered carbocycles or 5-7 membered heterocycles containing oxygen or sulfur or nitrogen.

3. The compound according to claim 2, wherein the compound of formula (I) has the following structure: 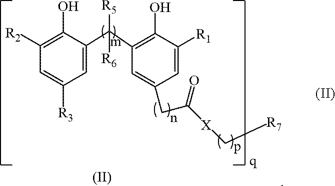
.

4. The compound according to claim 2, wherein the compound of formula (I) has the following structure: 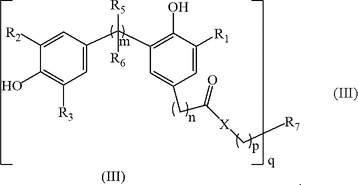
.

5. The compound of claim 4, wherein when R₇ is —(CH₂CH₂O)ₜCH₂CH₂—, t>1.

6. The compound according to claim 1, wherein R₁ and R₂ are independently selected from C₁-C₅-containing alkyl, phenyl, benzyl, and cumyl; R₃ and R₄ are independently selected from each other from hydrogen, hydroxyl, C₁~C₅ alkyl, acyl, C₁-C₅ alkylamino, C₁-C₅ alkoxy; R₅, R₆ are independently selected from hydrogen, hydroxyl, phenyl, C₁~C₅ alkyl, acyl group; R is selected from H, a bond, (C)ₐ(CH)_{b}(CH₂)_{c}(CH₃)_{d}, wherein a, b, c, d=0~18, a, b, c, d are not at the same time is 0, (CH₂CH₂O)ₜ H, (CH₂CH₂O)ₜOCH₃, (CH)_{q-2}(CH₂)_{0~12}(CH₃)_{1~3}, 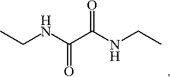 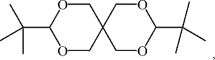
S, SH, O, OH, N, NH, NHR
₈, P, Ca, Mg, Zn, Na, K,-(CHR₈)_{1~18-}, -(CH)_{q-2}(CH₂)_{1~18-}, -(C=O)₁₋₄-, -(CHR₈)ᵤ (C=O)₁₋₄(CHR₈)ᵤ -, -(CHR₈)ᵤS₁₋₄(CHR₈)ᵤ-, -(CHR₈)ᵤO₁₋ ₄(CHR₈)ᵤ-, —(CH₂CH₂O)ₜCH₂CH₂—, triazines, melamines, unsubstituted or substituted phenyl or benzyl; q≧a+b+c+d ; t=1-20; u=1-10.

7. The compound according to claim 6, wherein, m=0-2; n=0-2; p=0-18; q=1-6; r=0-1; s=0-1; X=NH or O; R₇ is selected from H, a bond, C, CH, (CH₂)_{1~18}, CH₃, (CH₂CH₂O)_{1~8} H, (CH₂CH₂O)_{1~8}OCH₃, (CH)_{q-2}(CH₂)_{1~18}(CH₃)_{1~2}, 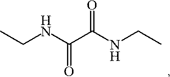 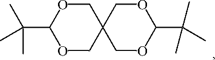
(S)
_{1∼2}, SH, O, OH, N, NH, NHR₈, P, Ca, Mg, Zn,—(C═0)₁₋₂-, -(CH₂)_{1~2} (CH)₁(CH₂)_{1~2}-, -(CHR₈)ᵤO₁₋₄(CHR₈)ᵤ-, triazine, melamine, phenyl, C_{1~4} alkyl substituted phenyl.

8. The compound according to claim 7, wherein, m=1; n=2; q=1-4; r=1; s=0.

9. The compound according to claim 8, selected from: 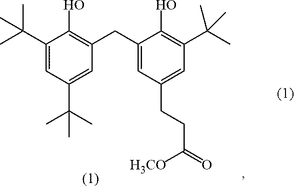 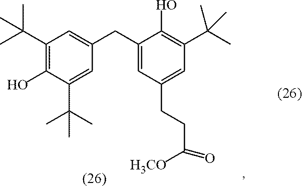 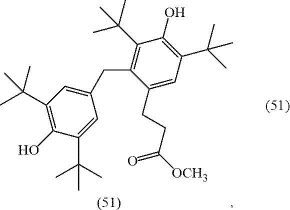 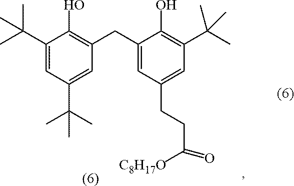 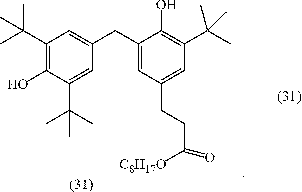 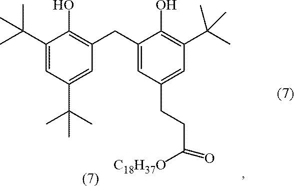 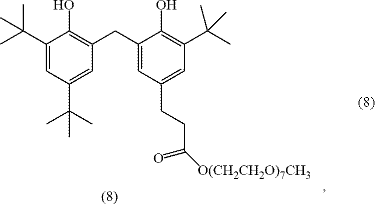 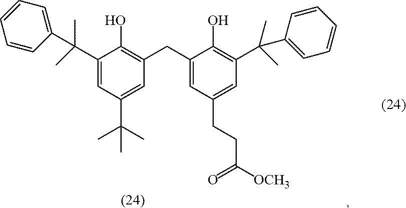 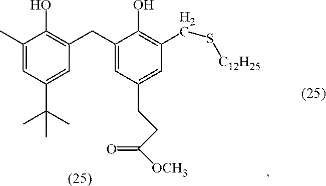 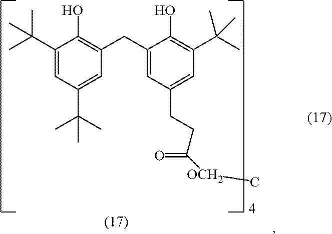 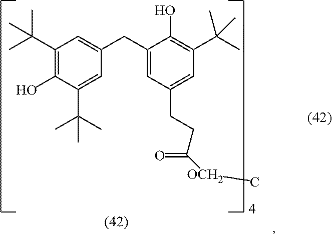 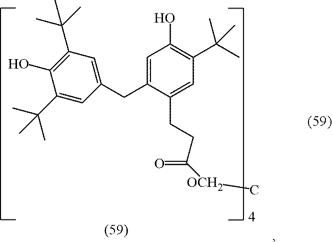 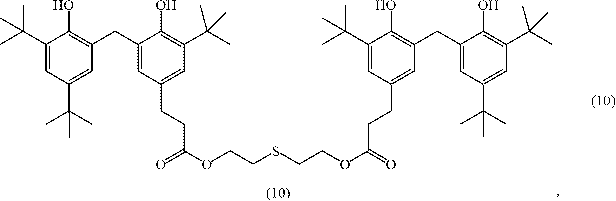 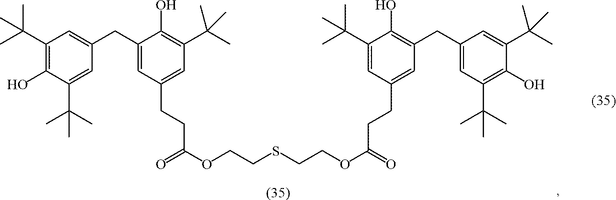 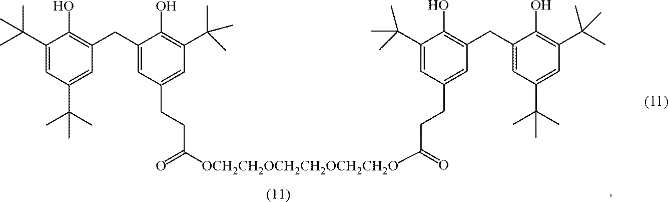 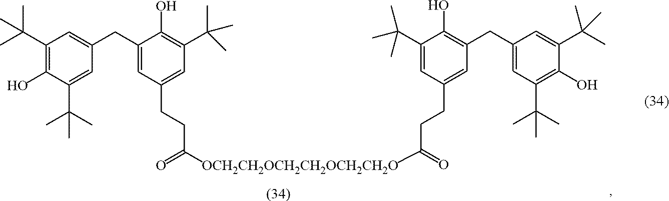 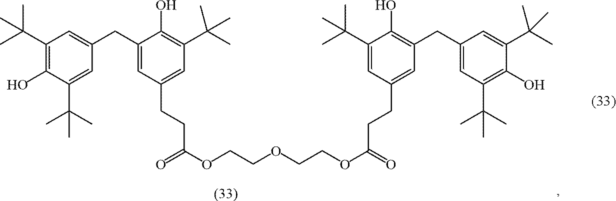 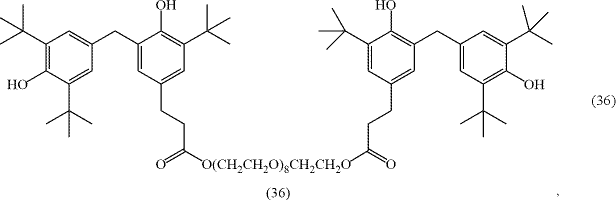 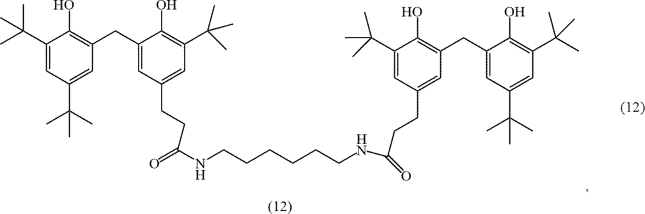 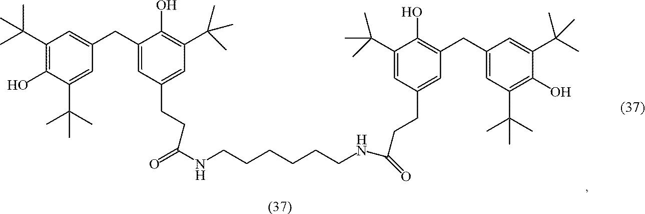 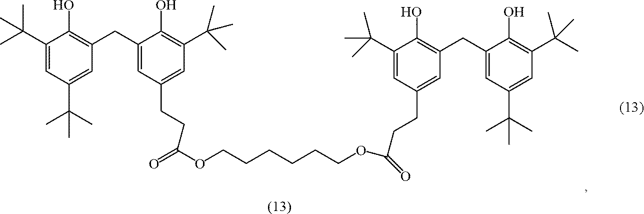 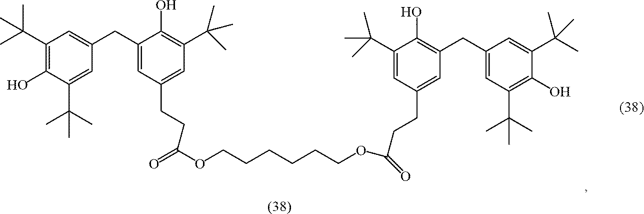 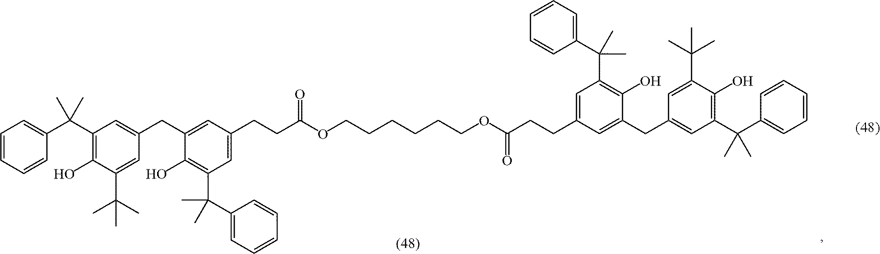 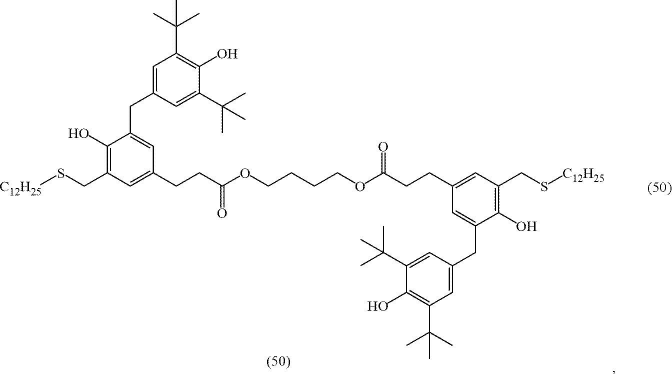 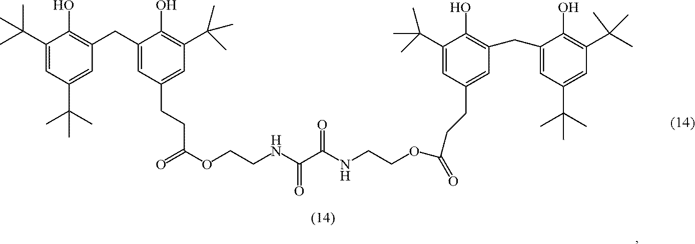 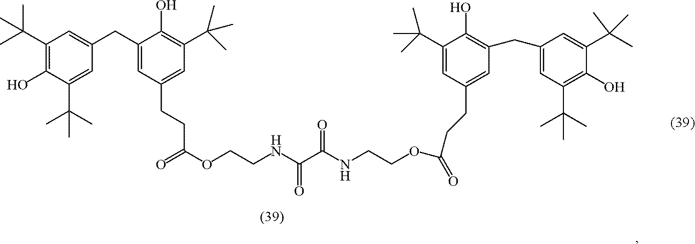 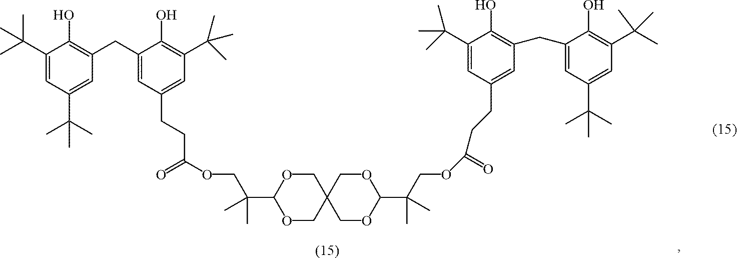 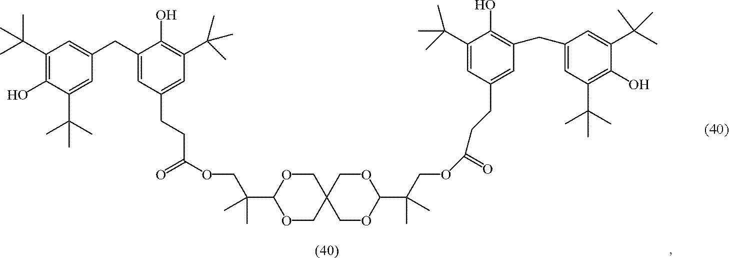 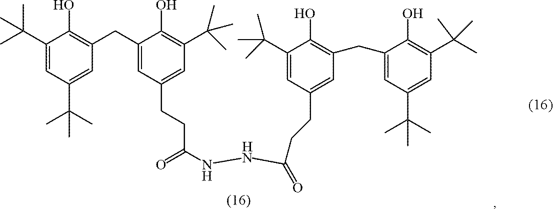 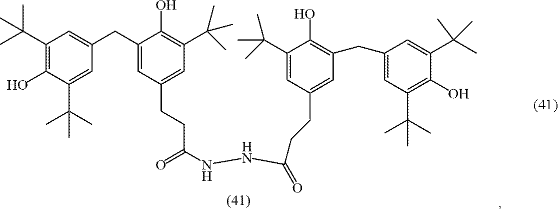 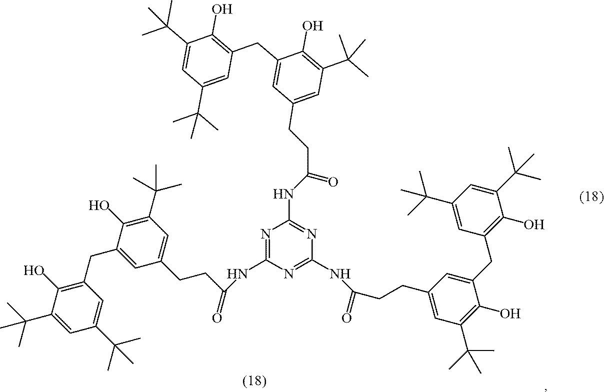 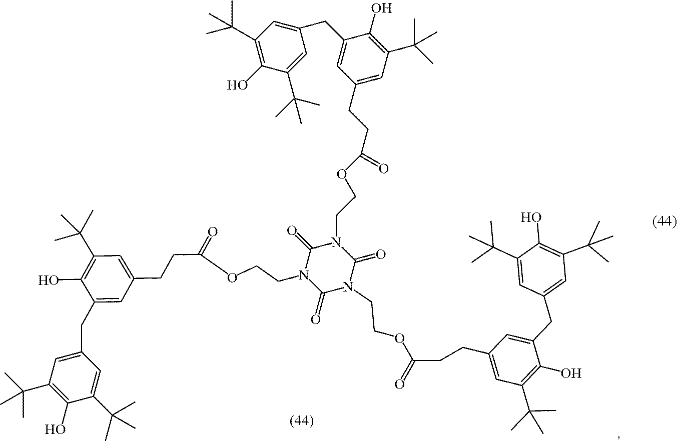 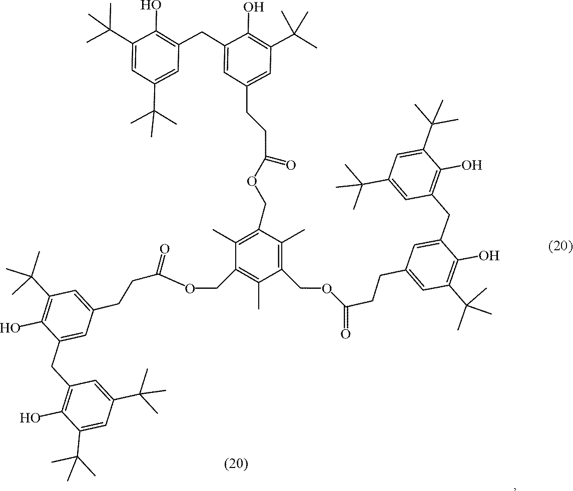 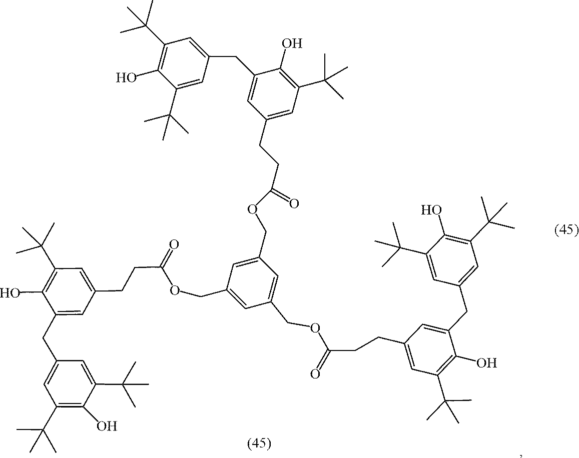 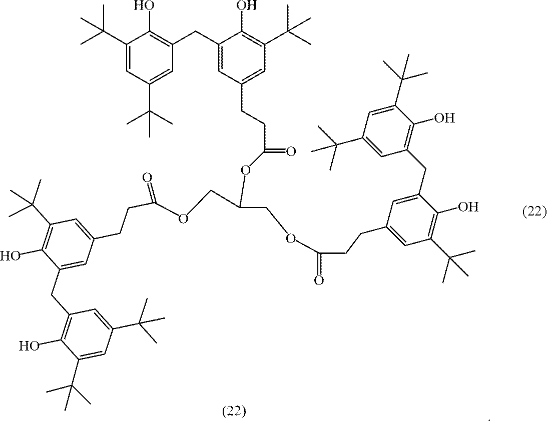
.

10. A method for preparation compound of formula (I), characterized in that, comprises following esterification or transesterification reaction, 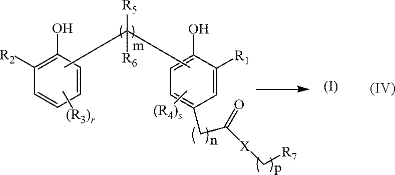
wherein R₁~R₇, n, X, p, r, s are as defined in claim 1; —X(CH₂)ₚR₇ is OH or a leaving group.

11. A composition comprising at least one compound of formula (I) or a salt thereof according to claim 1.

12. A method for preparing a hindered phenol antioxidant, comprises optimizing the ratio of (the number of hindered phenol units/molecular weight) of a known hindered phenol compound, so that the ratio of a new compound after optimization to the known compound is greater than 1.
